(19) United States
(12) Patent Application Publication
(10) Pub. No.: US 2008/0280943 A1
(43) Pub. Date: Nov. 13, 2008

(54) METHODS AND PACKAGES TO ENHANCE SAFETY WHEN USING IMIQUIMOD TO TREAT CHILDREN DIAGNOSED WITH SKIN DISORDERS

(76) Inventors: Herbert B. Slade, Woodbury, MN (US); James H. Lee, Devon, PA (US)

Correspondence Address: EDWARDS ANGELL PALMER & DODGE LLP, P.O. BOX 55874, BOSTON, MA, 02205, US

(21) Appl. No.: 12/054,357

(22) Filed: Mar. 24, 2008

Related U.S. Application Data
(60) Provisional application No. 60/896,792, filed on Mar. 23, 2007.
(60) Provisional application No. 60/896,805, filed on Mar. 23, 2007.
(60) Provisional application No. 60/896,811, filed on Mar. 23, 2007.
(60) Provisional application No. 60/896,815, filed on Mar. 23, 2007.
(60) Provisional application No. 60/896,817, filed on Mar. 23, 2007.
(60) Provisional application No. 60/896,830, filed on Mar. 23, 2007.
(60) Provisional application No. 60/896,835, filed on Mar. 23, 2007.
(60) Provisional application No. 60/896,838, filed on Mar. 23, 2007.
(60) Provisional application No. 60/896,844, filed on Mar. 23, 2007.
(60) Provisional application No. 60/896,846, filed on Mar. 23, 2007.
(60) Provisional application No. 60/896,850, filed on Mar. 23, 2007.
(60) Provisional application No. 60/896,870, filed on Mar. 23, 2007.
(60) Provisional application No. 60/896,871, filed on Mar. 23, 2007.
(60) Provisional application No. 60/896,873, filed on Mar. 23, 2007.
(60) Provisional application No. 60/896,875, filed on Mar. 23, 2007.
(60) Provisional application No. 60/896,876, filed on Mar. 23, 2007.
(60) Provisional application No. 60/896,879, filed on Mar. 23, 2007.
(60) Provisional application No. 60/896,881, filed on Mar. 23, 2007.
(60) Provisional application No. 60/896,882, filed on Mar. 23, 2007.
(60) Provisional application No. 60/896,885, filed on Mar. 23, 2007.
(60) Provisional application No. 60/896,887, filed on Mar. 23, 2007.
(60) Provisional application No. 60/896,889, filed on Mar. 23, 2007.
(60) Provisional application No. 60/896,890, filed on Mar. 23, 2007.
(60) Provisional application No. 60/896,891, filed on Mar. 23, 2007.
(60) Provisional application No. 60/896,892, filed on Mar. 23, 2007.

(51) Int. Cl. A61K 31/437; A61P 17/00

(52) U.S. Cl. 514/292

(57) ABSTRACT

Pharmaceutical packages and methods for enhancing the safety of imiquimod when used to treat children affected by skin disorders are disclosed. More particularly, the safety profile of imiquimod use is enhanced by providing information to the children, guardians of the children, including parents and health care professionals, that systemic absorption of imiquimod and other effects may be observed when imiquimod therapy is used to treat children of between about 2 and about 12 years of age. Examples of systemic absorption include a serum imiquimod concentrations of less than about 2 ng/mL, a decrease in median white blood cell count by about 1.4*10⁹/L or a decrease in median absolute neutrophil count by about 1.42*10⁹/L. Topical and/or transdermal delivery of imiquimod, including creams, ointments, gels, lotions, salves and pressure-sensitive adhesive compositions to treat dermatological disorders in children, namely, molluscum contagiosum, viral infections, such as Type I or Type II Herpes simplex infections and condyloma acuminata, genital warts and perianal warts, actinic keratosis and superficial basal cell carcinoma, and to induce interferon biosynthesis, are disclosed.

### CROSS-REFERENCE TO RELATED APPLICATIONS

This application claims priority under 35 U.S.C. 119(e) to U.S. Provisional Application Nos. 60/896,792; 60/896,805; 60/896,811; 60/896,815; 60/896,817; 60/896,830; 60/896,835; 60/896,838; 60/896,844; 60/896,846; 60/896,850; 60/896,870; 60/896,871; 60/896,873; 60/896,875; 60/896,876; 60/896,879; 60/896,881; 60/896,882; 60/896,885; 60/896,887; 60/896,889; 60/896,890; 60/896,891; 60/896,892, filed Mar. 23, 2007, the entirety of which is hereby incorporated by reference.

### FIELD OF THE INVENTION

This invention pertains to enhancing the safety of using pharmaceutical formulations containing 1-isobutyl-1H-imidazo[4,5-c]-quinolin-4-amine, i.e., imiquimod, to treat children diagnosed with skin disorders. More particularly, it pertains to methods and packages containing creams, ointments, foams, gels, lotions, salves, pressure sensitive adhesive coatings or adhesive-coated sheet materials, which contain imiquimod, that (i) enhance skin penetration of drugs to treat dermatological disorders, namely, molluscum contagiosum, viral infections, such as Type I or Type II Herpes simplex infections, e.g., condyloma acuminata, genital warts and perianal warts, actinic keratosis, and superficial basal cell carcinoma, and (ii) induce interferon biosynthesis, with enhanced safety by providing precautions and warnings that systemic absorption of imiquimod and other effects, namely, a decrease in median white blood cell counts or a decrease in median absolute neutrophil counts, may be observed when imiquimod therapy is used to treat children of between 2 and 12 years of age.

### BACKGROUND

The compound I-isobutyl-1H-imidazo[4,5-c]-quinolin-4-amine, known as imiquimod and commercially marketed in the U.S. under the brand name Aldara®, is disclosed in U.S. Pat. No. 4,689,338 and described therein as an antiviral agent and as an interferon inducer, which is incorporated herein by reference in its entirety. A variety of formulations for topical administration of imiquimod are also described therein. This U.S. Pat. No. 4,689,338 is incorporated herein by reference in its entirety.

U.S. Pat. No. 4,751,087 discloses the use of a combination of ethyl oleate and glyceryl monolaurate as a skin penetration enhancer for nitroglycerine, with all three components being contained in the adhesive layer of a transdermal patch, wherein this U.S. patent is incorporated herein by reference in its entirety.

U.S. Pat. No. 4,411,893 discloses the use of N,N-dimethyldodecylamine-N-oxide as a skin penetration enhancer in aqueous systems, wherein this U.S. patent is incorporated herein by reference in its entirety.

U.S. Pat. No. 4,722,941 discloses readily absorbable pharmaceutical compositions that comprise a pharmacologically active agent distributed in a vehicle comprising an absorption-enhancing amount of at least one fatty acid containing 6 to 12 carbon atoms and optionally a fatty acid monoglyceride. Such compositions are said to be particularly useful for increasing the absorption of pharmacologically active bases, wherein this U.S. patent is incorporated herein by reference in its entirety.

U.S. Pat. No. 4,746,515 discloses a method of using glyceryl monolaurate to enhance the transdermal flux of a transdermally deliverable drug through intact skin, wherein this U.S. patent is incorporated herein by reference in its entirety.

U.S. Pat. No. 5,238,944 discloses topical formulations and transdermal delivery systems containing 1-isobutyl-1H-imidazo[4,5-c]quinolin-4-amine, wherein this U.S. patent is incorporated herein by reference in its entirety.

The label or package insert for Aldara®, the only Food & Drug Administration (“FDA”) approved imiquimod product on the market in the United States, states that in pediatric use, (i) the safety and efficacy of Aldara Cream for external genital/perianal warts in patients below the age of 12 years have not been established, and (ii) the safety and efficacy of Aldara Cream for AK or sBCC in patients less than 18 years of age have not been established.

### SUMMARY OF THE INVENTION

In brief the present invention is directed to overcoming certain drawbacks and shortcomings associated with imiquimod therapy when treating children diagnosed with skin disorders, through the discovery of novel methods and packages containing topical imiquimod pharmaceutical products that enhance the safety of imiquimod when used to treat children.

In accordance with the present invention, it has been surprisingly discovered that systemic absorption of imiquimod may occur when treating children for a topical disorder. In addition, it has been surprisingly discovered that a reduction in median white blood cell counts and a reduction in median absolute neutrophil counts may also be observed while using topical imiquimod therapy.

Also in accordance with the present invention, it has been surprisingly discovered that the possible imiquimod systemic absorption in children when treated with topical imiquimod may manifest itself as, for example: (a) peak serum imiquimod concentrations in children between about 2 and about 0.12 years of age following both single and multiple doses at about <2 ng/ml; (b) median multiple-dose peak serum drug levels of approximately about 0.2 ng/ml or about 0.5 ng/ml in children ages from about 2 to about 5 years who receive imiquimod doses of about 12.5 mg (one packet) or about 25 mg (two packets), respectively; (c) median multiple dose serum drug levels of about 0.1 ng/ml, about 0.15 ng/ml, or about 0.3 ng/ml in children ages between about 6 and about 12 years who receive imiquimod doses of 12.5 mg, 25 mg, or 37.5 mg (three packets), respectively; (d) a median decrease in the white blood cell (“WBC”) count, such as a median WBC count decreased by about 1.4*109/L; and/or (e) a median decrease in the absolute neutrophil count, such as a median absolute neutrophil count decreased by about 1.42*109/L.

The present invention, therefore, provides methods and packages, to enhance the safety profile of imiquimod when used as topical therapy to treat children diagnosed with skin disorders, which comprises (i) providing information, such as to the child, a prescribing or treating physician, a treating nurse or guardian of the child, that systemic absorption of imiquimod may result in the child receiving imiquimod therapy, and (ii) providing further information to the child, the prescribing or treating physician, the treating nurse or the child's guardian, that the child should be monitored as safety and precautionary measures; namely, serum imiquimod levels, median white blood cell counts and median absolute neutrophil counts may be examined during the course of imiquimod therapy.

While it is believed that imiquimod is generally safe to use with children ages 2 to 12, the current invention nevertheless contemplates providing information to the children, the childrens' guardians, e.g., parents, the treating nurses, the prescribing/treating physicians or other health care officials, that consideration should be given to tapering or stopping imiquimod treatment, or reducing the imiquimod dose or frequency of imiquimod administration, in the event that (a) serum imiquimod concentrations exceed more than about 2 ng/mL, (b) there is a decrease in median white blood cell counts by at least about 1.4*109/L and/or (c) there is a decrease in median absolute neutrophil counts by at least about 1.42*109/L, until the symptoms have subsided, as safety and precautionary measures.

Examples of dermatological disorders contemplated by the present invention include (i) molluscum contagiosum possibly caused by a poxvirus of the (i) Molluscipox virus genus, (ii) viral infections, such as Type I or Type II Herpes simplex infections, e.g., condyloma acuminata, genital warts and perianal warts, actinic keratosis, and superficial basal cell carcinoma, and (iii) induce interferon biosynthesis. Examples of topical imiquimod formulations suitable for use in accordance with the present invention include creams, ointments, foams, gels, lotions, salves, pressure sensitive adhesive coatings and adhesive-coated sheet materials.

The present invention also provides a substantially non-irritating pharmaceutical formulation for topical and/or transdermal administration of the imiquimod, which formulation comprises:

a) 1-isobutyl-1H-imidazo[4,5-c]-quinolin-4-amine, i.e., imiquimod, in an amount of about 2 percent to about 4 percent by weight based on the total weight of the formulation; and
b) a pharmaceutically acceptable vehicle for imiquimod, which vehicle comprises a fatty acid, such as isostearic acid, linoleic acid, oleic acid, super purified oleic acid (an oleic acid having low polar impurities such as peroxides) and a combination thereof, in a total amount of about 3 percent to about 45 percent by weight based on the total weight of the formulation. The formulation is further characterized in that when tested in the hairless mouse skin model described in U.S. Pat. No. 5,238,944, the formulation provides a penetration of the agent of at least about 10% (and preferably at least about 15%) of the total amount of the agent contained in the formulation in 24 hours.

The salient elements of a pharmaceutical formulation according to the invention are (a) imiquimod and (b) a fatty acid, e.g., isostearic, linoleic, super purified oleic or oleic acid and mixtures thereof. A pharmaceutical formulation of the invention can be in any form known to the art, such as a cream, an ointment, a foam, a gel, a lotion or a pressure-sensitive adhesive composition, each form containing the necessary elements in particular amounts and further containing various additional elements.

A cream of the invention preferably contains about 2 percent to about 4 percent by weight of imiquimod based on the total weight of the cream; about 5 percent to about 25 percent by weight of fatty acid, based on the total weight of the cream; and optional ingredients such as emollients, emulsifiers, thickeners, and/or preservatives.

An ointment of the invention contains an ointment base in addition to imiquimod and fatty acid. An ointment of the invention preferably contains about 2 percent to about 4 percent by weight imiquimod; about 3 percent to about 45 percent, more preferably about 3 percent to about 25 percent by weight fatty acid; and about 40 percent to about 95 percent by weight ointment base, all weights being based on the total weight of the ointment. Optionally, an ointment of the invention can also contain emulsifiers, emollients and thickeners.

A pressure-sensitive adhesive composition of the invention contains imiquimod, fatty acid, and an adhesive. The adhesives utilized in a pressure sensitive adhesive composition of the invention are preferably substantially chemically inert to imiquimod. A pressure sensitive adhesive composition of the invention preferably contains about 2 percent to about 4 percent by weight imiquimod; about 10 percent to about 40 percent by weight, more preferably of about 15 percent to about 30 percent by weight, and most preferably about 20 percent to about 30 percent by weight of fatty acid; all weights being based on the total weight of the pressure sensitive adhesive composition.

Optionally, pressure sensitive adhesive compositions of the invention can also contain one or more skin penetration enhancers. The total amount of skin penetration enhancer(s) present in a pressure sensitive adhesive composition of the invention is preferably about 3 percent to about 25 percent by weight, and more preferably about 3 percent to about 10 percent by weight based on the total weight of the pressure sensitive adhesive composition.

A pressure sensitive adhesive coated sheet material of the invention can be made from a pressure-sensitive adhesive composition of the invention in the form of an article such as a tape, a patch, a sheet, or a dressing.

A formulation of the present invention may be used to topically and/or transdermally administer imiquimod for effectively treating viral infections, for example, Type I or Type II Herpes simplex infections, actinic keratosis and superficial basal cell carcinoma for a shorter duration of time and with the same or increased number of applications per week, as compared to current imiquimod topical therapy.

For example, a formulation of the present invention containing between greater than about 1% and about 5% imiquimod may be applied from three to seven times per week (once per day) for 8 to 12 weeks to treat viral infections, for example, Type I or Type II Herpes simplex infections, actinic keratosis and superficial basal cell carcinoma. It should be understood that while formulations of the present invention containing between greater than about 1% and about 5% imiquimod are preferred, formulations containing about 2.5%, 2.75%, 3.0%, 3.25%, 3.5%, 3.75%, 4.0%, 4.25% and 4.5% are more preferred and that formulations containing about 2.75%, 3.0%, 3.25%, 3.5%, 3.75% and 4.0% are most preferred.

As to duration, the present invention contemplates applying an effective amount of imiquimod for a shorter period of time than currently approved by the FDA. More specifically, the present invention contemplates applying an effective amount of imiquimod from three to seven times or more per week to an area in need of imiquimod treatment for about 8 to about 12 weeks, and more preferably between about 4, about 5, about 6 and about 7 times a week for about 8, about 9 or about 10 weeks.

While the present invention has identified what it believes to be preferred concentrations of imiquimod, numbers of applications per week and durations of therapy, it should be understood by those versed in this art that any effective concentration of imiquimod in a formulation and any numbers of application per week that can accomplish a reduction in therapy duration to effectively treat Type I or Type II Herpes simplex infections, actinic keratosis and superficial basal cell carcinoma or induce effective interferon biosynthesis is contemplated by the present invention.

### DETAILED DESCRIPTION OF THE INVENTION

As used in the specification and claims, the phrase “substantially non-irritating” designates formulations that do not cause unacceptable skin irritation in conventional repeat skin irritation tests in albino rabbits such as that described in Draize et al., “Appraisal of the Safety of Chemicals in Food, Drugs and Cosmetics”, prepared by the Division of Pharmacology of the Food and Drug Administration, published originally in 1959 by the Association of Food and Drug Officials of the United States, Topeka, Kans. (2nd printing 1965), incorporated herein by reference.

The present invention provides pharmaceutical formulations such as creams, ointments, foams, gels, lotions and adhesive coatings that contain imiquimod and a fatty acid such as isostearic, linoleic, super purified oleic acid or oleic acid and mixtures thereof. The formulations of the invention provide desirable skin penetrability of the imiquimod.

The compound imiquimod is a known antiviral agent that is also known to induce interferon biosynthesis. It can be prepared using the method disclosed in U.S. Pat. No. 4,689,338, the disclosure of which is incorporated herein by reference. The compound can be used to treat viral infections such as Type I or Type II Herpes simplex infections and genital warts. Furthermore, the fact that the compound is an interferon inducer suggests that it, and therefore formulations containing it, might be useful in the treatment of numerous other diseases, such as rheumatoid arthritis, warts, eczema, hepatitis B, psoriasis, multiple sclerosis, essential thrombocythaemia, and cancer, such as basal cell carcinoma and other neoplastic diseases. The amount of imiquimod present in a formulation of the invention will be an amount effective to treat the targeted disease state to prevent the recurrence of such a disease or to promote immunity against such a disease. The amount is preferably about 0.5 percent to about 9 percent by weight based on the total weight of a formulation, more preferably between greater than about 1% and about 5% imiquimod, and more preferably between about 2.5%, 2.75%, 3.0%, 3.25%, 3.5%, 3.75%, 4.0%, 4.25% and 4.5%, and most preferred between about 2.75%, 3.0%, 3.25%, 3.5%, 3.75% and 4.0%.

A fatty acid such as isostearic acid, linoleic acid, super purified oleic acid, oleic acid or a mixture thereof is incorporated into a formulation of the invention. The total amount of fatty acid present in a formulation is preferably about 3 percent to about 45 percent by weight based on the total weight of a formulation. It should be understood that when oleic acid is selected as a fatty acid, that stability may present issue. Thus, stabilizers, such as anti-oxidants and the like may be required to preserve pharmaceutical elegance and stability over the life of the oleic formulation.

A pharmaceutical formulation of the invention can be in a form such as a cream, an ointment, a foam, a gel, a lotion, a pressure-sensitive adhesive composition, or other forms known to those skilled in the art, each particular form containing imiquimod and fatty acid in particular amounts, and optionally containing various additional elements. The preferred amounts of drug and fatty acid, and the amounts and types of optional elements used in formulations of the invention are discussed below with particular reference to creams, ointments and adhesive compositions.

A cream according to the invention contains 1-isobutyl-1H-imidazo[4,5-c]quinolin-4-amine and fatty acid.

The amount of 1-isobutyl-1H-imidazo[4,5-c]-quinolin-4-amine present in a cream is preferably about 0.5 percent to about 9 percent by weight, and more preferably about 1 percent to about 5 percent by weight, based on the total weight of the cream.

The total amount of fatty acid present in a cream of the invention is preferably about 3 percent to about 45 percent by weight, and more preferably about 5 percent to about 25 percent by weight, based on the total weight of the cream.

Optionally, a cream of the invention can contain emollients, emulsifiers, thickeners, and/or preservatives.

Emollients such as long chain alcohols e.g., cetyl alcohol, stearyl alcohol and cetearyl alcohol; hydrocarbons such as petrolatum and light mineral oil; or acetylated lanolin can be included in a cream of the invention. A cream can contain one or more of these emollients. The total amount of emollient in a cream of the invention is preferably about 5 percent to about 30 percent, and more preferably about 5 percent to about 10 percent by weight based on the total weight of the cream.

Emulsifiers such as nonionic surface active agents, e.g., polysorbate 60 (available from ICI Americas), sorbitan monostearate, polyglyceryl-4 oleate, and polyoxyethylene(4)lauryl ether or trivalent cationic a cream of the invention. A cream can contain one or more emulsifiers. Generally the total amount of emulsifier is preferably about 2 percent to about 14 percent, and more preferably about 2 percent to about 6 percent by weight based on the total weight of the cream.

Pharmaceutically acceptable thickeners, such as Veegum™K (available from R. T. Vanderbilt Company, Inc.), and long chain alcohols (i.e. cetyl alcohol, stearyl alcohol or cetearyl alcohol) can be used. A cream can contain one or more thickeners. The total amount of thickener present is preferably about 3 percent to about 12 percent by weight based on the total weight of the cream.

Preservatives such as methylparaben, propylparaben and benzyl alcohol can be present in a cream of the invention. The appropriate amount of such preservative(s) is known to those skilled in the art.

Optionally, an additional solubilizing agent such as benzyl alcohol, lactic acid, acetic acid, stearic acid or hydrochloric acid can be included in a cream of the invention.

If an additional solubilizing agent is used, the amount present is preferably about 1 percent to about 12 percent by weight based on the total weight of the cream.

Optionally, a cream of the invention can contain a humectant such as glycerin, skin penetration enhancers such as butyl stearate, and additional solubilizing agents.

It is known to those skilled in the art that a single ingredient can perform more than one function in a cream, i.e., cetyl alcohol can serve both as an emollient and as a thickener.

Generally, a cream consists of an oil phase and a water phase mixed together to form an emulsion. Preferably, the amount of water present in a cream of the invention is about 45 percent to about 85 percent by weight based on the total weight of the cream.

The oil phase of a cream of the invention can be prepared by first combining the 1-isobutyl-1H-imidazo[4,5-c]quinolin-4-amine and the fatty acid (if the cream contains benzyl alcohol it can also be added at this point) and heating with occasional stirring to a temperature of about 50° C. to 85° C. When the 1-isobutyl-1H-imidazo[4,5-c]quinolin-4-amine appears to be completely dissolved, the remaining oil phase ingredients are added and heating is continued until dissolution appears to be complete.

The water phase can be prepared by combining all other ingredients and heating with stirring until dissolution appears to be complete.

The creams of the invention are generally prepared by adding the water phase to the oil phase with both phases at a temperature of about 65° C. to 75° C. The resulting emulsion is mixed with a suitable mixer apparatus to give the desired cream.

An ointment of the invention contains an ointment base in addition to 1-isobutyl-1H-imidazo[4,5-c]quinolin-4-amine and fatty acid.

The amount of 1-isobutyl-1H-imidazo[4,5-c]-quinolin-4-amine present in an ointment of the invention is preferably about 0.5 percent to about 9 percent, and more preferably about 0.5 percent to about 5 percent by weight based on the total weight of the ointment.

The total amount of fatty acid present in an ointment of the invention is preferably about 3 percent to about 45 percent, and more preferably about 3 percent to about 25 percent based on the total weight of the ointment.

A pharmaceutically acceptable ointment base such as petrolatum or polyethylene glycol 400 (available from Union Carbide) in combination with polyethylene glycol 3350 (available from Union Carbide) can be used. The amount of ointment base present in an ointment of the invention is preferably about 60 percent to about 95 percent by weight based on the total weight of ointment.

Optionally, an ointment of the invention can also contain emollients, emulsifiers and thickeners. The emollients, emulsifiers, and thickeners and the preferred amounts thereof described above in connection with creams are also generally suitable for use in an ointment of the invention.

An ointment according to the invention can be prepared by combining 1-isobutyl-1H-imidazo[4,5-c]quinolin-4-amine with fatty acid and heating with occasional stirring to a temperature of about 65° C. When the 1-isobutyl-1H-imidazo[4,5-c]-quinolin-4-amine appears to be completely dissolved, the remaining ingredients are added and heated to about 65° C. The resulting mixture is mixed with a suitable mixer while being allowed to cool to room temperature.

A pressure-sensitive adhesive composition of the invention contains 1-isobutyl-1H-imidazo[4,5-c]-quinolin-4-amine, fatty acid, and a pressure sensitive adhesive polymer.

The amount of 1-isobutyl-1H-imidazo[4,5-c]-quinolin-4-amine present in a pressure sensitive adhesive composition of the invention is preferably about 0.5 percent to about 9 percent by weight, and more preferably about 3 percent to about 7 percent by weight based on the total weight of the adhesive composition. The amount of fatty acid present is preferably about 10 percent to about 40 percent by weight, more preferably about 15 percent to about 30 percent by weight, and most preferably about 20 percent to about 30 percent by weight, based on the total weight of the adhesive composition.

Preferably, the adhesive polymer utilized in a pressure sensitive adhesive composition of the invention is substantially chemically inert to 1-isobutyl-1H-imidazo[4,5-c]quinolin-4-amine. The adhesive polymer is preferably present in an amount of about 55 percent to about 85 percent by weight based on the total weight of the composition. Suitable adhesive polymers include acrylic adhesives that contain, as a major constituent (i.e., at least about 80 percent by weight of all monomers in the polymer), a hydrophobic monomeric acrylic or methacrylic acid ester of an alkyl alcohol, the alkyl alcohol containing 4 to 10 carbon atoms. Examples of suitable monomers are those discussed below in connection with the “A Monomer”. These adhesive polymers can further contain minor amounts of other monomers such as the “B Monomers” listed below.

Preferred adhesives include acrylic pressure-sensitive adhesive copolymers containing A and B Monomers as follows: Monomer A is a hydrophobic monomeric acrylic or methacrylic acid ester of an alkyl alcohol, the alkyl alcohol containing 4 to 10 carbon atoms, preferably 6 to 10 carbon atoms, more preferably 6 to 8 carbon atoms, and most preferably 8 carbon atoms. Examples of suitable A Monomers are n-butyl, n-pentyl, n-hexyl, isoheptyl, n-nonyl, n-decyl, isohexyl, 2-ethyloctyl, isooctyl and 2-ethylhexyl acrylates. The most preferred A Monomer is isooctyl acrylate.

Monomer B is a reinforcing monomer selected from the group consisting of acrylic acid; methacrylic acid; alkyl acrylates and methacrylates containing 1 to 3 carbon atoms in the alkyl group; acrylamide; methacrylamide; lower alkyl-substituted acrylamides (i.e., the alkyl group containing 1 to 4 carbon atoms) such as tertiary-butyl acrylamide; diacetone acrylamide; n-vinyl-2-pyrrolidone; vinyl ethers such as vinyl tertiary-butyl ether; substituted ethylenes such as derivatives of maleic anhydride, dimethyl itaconate and monoethyl formate and vinyl perfluoro-n-butyrate. The preferred B Monomers are acrylic acid, methacrylic acid, the above-described alkyl acrylates and methacrylates, acrylamide, methacrylamide, and the above-described lower alkyl substituted acrylamides. The most preferred B Monomer is acrylamide.

In one embodiment of a pressure-sensitive adhesive composition of the invention, the pressure-sensitive adhesive copolymer containing A and B Monomers as set forth above preferably contains the A Monomer in an amount by weight of about 80 percent to about 98 percent of the total weight of all monomers in the copolymer. The A Monomer is more preferably present in an amount by weight of about 88 percent to about 98 percent, and is most preferably present in an amount by weight of about 91 percent to about 98 percent. The B Monomer in such a copolymer is preferably present in the pressure-sensitive adhesive copolymer in an amount by weight of about 2 percent to about 20 percent, more preferably about 2 percent to about 12 percent, and most preferably 2 to 9 percent of the total weight of the monomers in the copolymer.

In another embodiment of a pressure-sensitive adhesive composition of the invention, the adhesive copolymer comprises about 60 to about 80 percent by weight (and preferably about 70 to about 80 percent by weight) of the above-mentioned hydrophobic monomeric acrylic or methacrylic acid ester of an alkyl alcohol (i.e., Monomer A described above) based on the total weight of all monomers in the copolymer; about 4 to about 9 percent by weight based on the total weight of all monomers in the copolymer of a reinforcing monomer selected from the group consisting of acrylic acid, methacrylic acid, an alkyl acrylate or methacrylate containing 1 to 3 carbon atoms in the alkyl group, acrylamide, methacrylamide, a lower alkyl-substituted acrylamide, diacetone acrylamide and N-vinyl-2-pyrrolidone; and about 15 to about 35 percent by weight (and preferably about 15 to about 25 percent by weight) of vinyl acetate based on the total weight of all monomers in the copolymer. In this embodiment the preferred acrylic or methacrylic acid ester is isooctyl acrylate and the preferred reinforcing monomer is acrylamide.

The above described adhesive copolymers are known, and methods of preparation therefor are well known to those skilled in the art, having been described for example, in U.S. Pat. No. 24,906 (Ulrich), the disclosure of which is incorporated herein by reference. The polymerization reaction can be carried out using a free radical initiator such as an organic peroxide (e.g., benzoylperoxide) or an organic azo compound (e.g., 2,2′-azobis(2,4-dimethylpentanenitrile), available under the trade designation “Vazo 52” from DuPont).

Since pressure-sensitive adhesives such as those described above are inherently rubbery and tacky and are suitably heat and light stable, there is no need to add tackifiers or stabilizers. However, such can be added if desired.

Optionally, a pressure sensitive adhesive composition of the invention can also contain one or more skin penetration enhancers such as glyceryl monolaurate, ethyl oleate, isopropyl myristate, diisopropyl adipate and N,N-dimethyldodecylamine-N-oxide, either as a single ingredient or as a combination of two or more ingredients. The skin penetration enhancer(s) preferably form a substantially homogeneous mixture with the pressure sensitive adhesive polymer or copolymer. The total amount of skin penetration enhancer(s) present in a pressure sensitive adhesive composition of the invention is preferably about 3 percent to about 25 percent by weight, more preferably about 3 percent to about 10 percent by weight based on the total weight of the adhesive composition.

When the skin penetration enhancer is a single ingredient, it is preferably a skin penetration enhancer such as isopropyl myristate, diisopropyl adipate, ethyl oleate, or glyceryl monolaurate.

When a combination skin penetration enhancer is used, it is preferably a combination such as: ethyl oleate with glyceryl monolaurate; ethyl oleate with N,N-dimethyldodecylamine-N-oxide; glyceryl monolaurate with N,N-dimethyldodecylamine-N-oxide; and ethyl oleate with both glyceryl monolaurate and N,N-dimethyldodecylamine-N-oxide.

A pressure-sensitive adhesive composition of the invention can be prepared by combining dry adhesive, 1-isobutyl-1H-imidazo[4,5-c]quinolin-4-amine, fatty acid, and skin penetration enhancer(s) with an organic solvent. The preferred organic solvents are methanol and ethyl acetate. The total solids content of the adhesive coating is preferably in the range of about 15 percent to about 40 percent, and more preferably in the range of about 20 to about 35 percent based on the total weight of the adhesive coating. The resulting mixture is shaken or mixed for a period of about 20 to 72 hours. When this method is used it is preferred that the 1-isobutyl-1H-imidazo[4,5-c]-quinolin-4-amine be in micronized form (i.e., particle size of 1-2 microns in diameter). Optionally, the mixture can be heated during shaking.

In a preferred method, the 1-isobutyl-1H-imidazo-4,5-c]quinolin-4-amine is combined with the fatty acid and shaken at 40° C. until there appears to be complete dissolution. The remaining ingredients are added and the mixture is shaken for a period of about 20 to 72 hours.

The pressure-sensitive adhesive compositions described above are preferably coated onto one surface of a suitable backing of sheet material, such as a film, to form a pressure-sensitive adhesive coated sheet material. A pressure-sensitive adhesive coated sheet material of the invention can be prepared by knife coating a suitable release liner to a predetermined uniform thickness with a wet adhesive formulation. This adhesive coated release liner is then dried and laminated onto a backing using conventional methods. Suitable release liners include conventional release liners comprising a known sheet material, such as a polyester web, a polyethylene web, or a polystyrene web, or polyethylene-coated paper, coated with a suitable silicone-type coating such as that available under the trade designation Daubert 164Z, from Daubert Co. The backing can be occlusive, non-occlusive or a breathable film as desired. The backing can be any of the conventional materials for pressure-sensitive adhesive tapes, such as polyethylene, particularly low density polyethylene, linear low density polyethylene, high density polyethylene, randomly-oriented nylon fibers, polypropylene, ethylene-vinylacetate copolymer, polyurethane, rayon and the like. Backings that are layered, such as polyethylene-aluminum-polyethylene composites are also suitable. The backing should be substantially non-reactive with the ingredients of the adhesive coating. The presently preferred backing is low density polyethylene.

The pressure-sensitive adhesive coated sheet material of the invention can be made in the form of an article such as a tape, a patch, a sheet, a dressing or any other form known to those skilled in the art.

Preferably, an article in the form of a patch is made from an adhesive coated sheet material of the invention and applied to the skin of a mammal. The patch is replaced as necessary with a fresh patch to maintain the particular desired therapeutic effect of the 1-isobutyl-1H-imidazo[4,5-c]quinolin-4-amine.

### Inherent Viscosity Measurement

The inherent viscosity values reported in the Examples below were obtained by the conventional method used by those skilled in the art. The measurement of the viscosity of dilute solutions of the adhesive, when compared to controls run under the same conditions, clearly demonstrates the relative molecular weights. It is the comparative values that are significant; absolute figures are not required. In the examples, the inherent viscosity values were obtained using a Cannon-Fenske #50 viscometer to measure the flow time of 10 ml of a polymer solution (0.2 g polymer/deciliter tetrahydrofuran, in a water bath controlled at 25° C.). The examples and the controls were run under identical conditions. The test procedure followed and the apparatus used are explained in detail in the Textbook of Polymer Science, F. W. Billmeyer, Wiley-Interscience, 2nd Edition, 1971 under: Polymer chains and their characterization, D. Solution Viscosity and Molecular Size, pp 84-85, the disclosure and textbook of which is incorporated by reference.

The following examples are provided to illustrate the invention, but are not intended to be limiting thereof. Parts and percentages are by weight unless otherwise specified. Examples of creams, ointments and pressure sensitive adhesive compositions contemplated by the present invention are described in U.S. Pat. No. 4,689,338 and U.S. Pat. No. 5,238,944. Percent modifications for, e.g., imiquimod and vehicle, to generate imiquimod formulations as described herein are likewise contemplated by the present invention. In addition, the formulations described and disclosed in U.S. patent application Ser. No. 11/276,324, are also contemplated by the present invention. Thus, U.S. patent application Ser. No. 11/276,324, is incorporated herein by reference in its entirety.

### Preparative Method 1

### Laboratory Scale Preparation of Isooctylacrylate/Acrylamide Copolymer

To a 114 gram narrow-mouth glass bottle were added: 18.6 g isooctyl acrylate, 1.4 g acrylamide, 0.04 g benzoyl peroxide, 27.0 g ethyl acetate and 3.0 g methanol. The solution was purged for thirty five seconds with nitrogen at a flow rate of one liter per minute. The bottle was sealed and placed in a rotating water bath at 55° C. for twenty-four hours to effect essentially complete polymerization. The polymer was diluted with ethyl acetate/methanol (90/10) to 23.2 percent solids and had a measured inherent viscosity of 1.26 dl/g in ethyl acetate.

### Preparative Method 2

### Pilot Plant Scale Preparation of Isooctylacrylate/Acrylamide Copolymer

155 kg isooctylacrylate, 11.6 kg acrylamide, 209.1 kg ethyl acetate and 23.2 kg methanol were charged to a clean, dry reactor. Medium agitation was applied. The batch was deoxygenated with nitrogen while heating to an induction temperature of 55° C. 114 g Lucidol™70 initiator (available from Pennwalt Corp.) mixed with 2.3 kg ethyl acetate was charged to the reactor. The temperature was maintained at 55° C. throughout the reaction. After 5.5 hours reaction time, 114 g Lucidol™70 mixed with 2.3 kg ethyl acetate were charged to the reactor. After 9.0 hours reaction time, an additional 114 g Lucidol™70 initiator mixed with 2.3 kg ethyl acetate were charged to the reactor. The reaction was continued until the percent conversion was greater than 98 percent as measured by gas chromatographic evaluation of residual monomer concentration. The resulting polymer solution was diluted to 25-28 percent solids with ethyl acetate/methanol (90/10) and had a measured Brookfield viscosity of 17,000-21,000 centipoises using spindle #4 at 12 rpm. The polymer had a measured inherent viscosity of 1.3-1.4 dl/g in ethyl acetate.

The above procedure was found to provide a pressure-sensitive adhesive that is equivalent in the practice of the present invention to a pressure-sensitive adhesive prepared according to PREPARATIVE METHOD 1.

A 25-30 percent solids solution of the isooctyl acrylate:acrylamide (93:7) adhesive copolymer in ethyl acetate/methanol (90:10) was coated onto a two-sided release liner using a knife-coater and coating at 0.5 mm in thickness. The adhesive-coated laminate was dried first at 82° C. for 3 minutes and then at 116° C. for 3 minutes. The dried adhesive coating was then stripped off the release liner and placed in a glass bottle. The foregoing procedure results in a reduction of the amount of any residual monomer in the adhesive copolymer.

### Preparative Method 3

### Preparation of Isooctyl Acrylate:Acrylamide:Vinyl Acetate (75:5:20) Copolymer

The procedure of PREPARATIVE METHOD 1 above acrylate, 8.0 g acrylamide, 32.0 g vinyl acetate, 0.32 g benzoyl peroxide, 216.0 g ethyl acetate and 24.0 g methyl alcohol. The resulting polymer was diluted with the ethyl acetate/methyl alcohol mixture to 21.52% solids. The adhesive polymer had a measured inherent viscosity of 1.40 dl/g in ethyl acetate at a concentration of 0.15 g/dl. Its Brookfield viscosity was 2,300 centipoise.

### Preparative Method 4

### Preparation of Isooctyl Acrylate Acrylamide:Vinyl Acetate (75:5:20) Copolymer

A master batch was prepared by combining 621.0 g of isooctyl acrylate, 41.4 g of acrylamide, 165.6 g of vinyl acetate, 1.656 g of 2,2′-azobis(2,4-dimethylpentanenitrile) (available from the DuPont Company as Vazo™52), 884.52 g of ethyl acetate and 87.48 g of methanol. A 400 g portion of the resulting solution was placed in an amber quart bottle. The bottle was purged for two minutes with nitrogen at a flow rate of one liter per minute. The bottle was sealed and placed in a rotating water bath at 45° C. for twenty-four hours to effect essentially complete polymerization. The copolymer was diluted with 250 g of ethyl acetate/methanol (90/10) to 26.05% solids and had a measured inherent viscosity of 1.27 dl/g in ethyl acetate at a concentration of 0.15 g/dl. Its Brookfield viscosity was 5580 centipoise.

### EXAMPLE 1

A cream according to the present invention was prepared from the following ingredients:

| | % by Weight | Amount | |
|---|---|---|---|
| Oil Phase | | | |
| 1-Isobutyl-1H-imidazo[4,5-c]- | 1.0 | 40.0 | g |
| quinolin-4-amine Isostearic acid | 10.0 | 400.0 | g |
| Benzyl alcohol | 2.0 | 80.0 | g |
| Cetyl alcohol | 2.2 | 88.0 | g |
| Stearyl alcohol | 3.1 | 124.0 | g |
| Polysorbate 60 | 2.55 | 102.0 | g |
| Sorbitan monostearate | 0.45 | 18.0 | g |
| Aqueous Phase | | | |
| Glycerin | 2.0 | 80.0 | g |
| Methylparaben | .02 | 8.0 | g |
| Propylparaben | 0.02 | .8 | g |
| Purified water | 76.48 | 3059.2 | g |

The materials listed above were combined according to the following procedure:

The glycerin, methylparaben, propylparaben and water were weighed into a 4 liter glass beaker then heated on a hot plate with stirring until the parabens isostearic acid and 1-isobutyl-1H-imidazo[4,5-c]-quinolin-4-amine were weighed into an 8 liter stainless steel beaker and heated on a hot plate until the amine was in solution (the temperature reached 69° C.). The benzyl alcohol, cetyl alcohol, stearyl alcohol, polysorbate 60 and sorbitan monostearate were added to the isostearic acid solution and heated on a hot plate until all material was dissolved (the temperature reached 75° C.). With both phases at approximately the same temperature (65°-75° C.), the water phase was added to the oil phase. The mixture was mixed with a homogenizer for 13 minutes then put into a cool water bath and mixed with a 3 inch propeller for 40 minutes (the temperature was 29° C.). The resulting cream was placed in glass jars.

### EXAMPLES 2-9

Using the general method of Example 1, the cream formulations shown in Tables 1 and 2 were prepared.

TABLE 1

% by Weight
Example

| | 2 | 3 | 4 | 5 |
|---|---|---|---|---|
| Oil Phase | | | | |
| 1-Isobutyl-1H-imidazo- | 1.0 | 1.0 | 1.0 | 1.0 |
| [4,5-c]quinolin-4-amine | | | | |
| Isostearic acid | 10.0 | 10.0 | 5.0 | 5.0 |
| Benzyl alcohol | — | 2.0 | — | — |
| Cetyl alcohol | — | 1.7 | — | — |
| Stearyl alcohol | — | 2.3 | — | — |
| Cetearyl alcohol | 6.0 | — | 6.0 | 6.0 |
| Polysorbate 60 | 2.55 | 2.55 | 2.55 | 2.55 |
| Sorbitan monostearate | 0.45 | 0.45 | 0.45 | 0.45 |
| Brij .TM. 30^{a} | — | — | — | 10.0 |
| Aqueous Phase | | | | |
| Glycerin | 2.0 | 2.0 | 2.0 | 2.0 |
| Methylparaben | 0.2 | 0.2 | 0.2 | 0.2 |
| Propylparaben | 0.02 | 0.02 | 0.02 | 0.02 |
| Purified water | 77.78 | 77.78 | 82.78 | 72.78 |

Brij .TM. 30 (polyoxyethylene(4) lauryl ether) is available from ICI Americas, Inc.

TABLE 2

| | % by Weight | | | |
|---|---|---|---|---|
| | Example | | | |
| | 6 | 7 | 8 | 9 |
| Oil Phase | | | | |
| 1-Isobutyl-1H-imidazo- | 1.0 | 1.0 | 1.0 | 1.0 |
| [4,5-c]quinolin-4-amine Isostearic acid | 10.0 | 25.0 | 10.0 | 6.0 |
| Benzyl alcohol | — | 2.0 | — | 2.0 |
| Cetyl alcohol | — | 2.2 | 1.7 | — |
| Stearyl alcohol | — | 3.1 | 2.3 | — |
| Cetearyl alcohol | 6.0 | — | — | 6.0 |
| Polysorbate 60 | 2.55 | 3.4 | 2.55 | 2.55 |
| Sorbitan monostearate | 0.45 | 0.6 | 0.45 | 0.45 |
| Brij .TM. 30 | 10.0 | — | — | — |
| Aqueous Phase | | | | |
| Glycerin | 2.0 | 2.0 | 2.0 | 2.0 |
| Methylparaben | 0.2 | 0.2 | 0.2 | 0.2 |
| Propylparaben | 0.02 | 0.02 | 0.02 | 0.02 |
| Purified water | 67.78 | 60.48 | 79.78 | 79.78 |

### EXAMPLE 10

A cream according to the present invention was prepared from the following ingredients.

| | % by Weight | Amount | |
|---|---|---|---|
| Oil Phase | | | |
| 1-Isobutyl-1H-imidazo[4,5-c]- | 1.0 | 3.00 | g |
| quinolin-4-amine | | | |
| Isostearic acid | 5.0 | 15.0 | g |
| White petrolatum | 15.0 | 45.0 | g |
| Light mineral oil | 12.8 | 38.4 | g |
| Aluminum stearate | 8.0 | 24.0 | g |
| Cetyl alcohol | 4.0 | 12.0 | g |
| Witconol .TM. 14^{a} | 3.0 | 9.00 | g |
| Acetylated lanolin | 1.0 | 3.0 | g |
| Propylparaben | 0.063 | 0.19 | g |
| Aqueous Phase | | | |
| Veegum .TM. K^{b} | 1.0 | 3.0 | g |
| Methylparaben | 0.12 | 0.36 | g |
| Purified water | 49.017 | 147.05 | g |

^{a}Witconol .TM. 14 (polyglyceryl4 oleate) is available from Witco Chemical Corp. Organics Division
^{b}Veegum .TM. K (colloidal magnesium aluminum silicate) is available from R. T. Vanderbilt Company Inc.

The materials listed above were combined according to the following procedure:

The 1-isobutyl-1H-imidazo[4,5-c]quinolin-4-amine and theisostearic acid were weighed into a glass jar and heated with occasional stirring until the amine was dissolved (the temperature reached 68° C.). To this solution was added, the petrolatum, mineral oil, aluminum stearate, cetyl alcohol, Witconol™14, acetylated lanoline and propylparaben. The mixture was heated to 75° C. In a separate beaker, the methylparaben and water were combined and heated until the paraben dissolved (the temperature reached 61° C.). The Veegum™K was added to the aqueous solution and heated at 75° C. for 30 minutes while mixing with a homogenizer. With both phases at 75° C., the aqueous phase was slowly added to the oil phase while mixing with a homogenizer. Mixing was continued for 30 minutes while maintaining a temperature to about 80° C. The jar was then capped and the formulation was allowed to cool.

### EXAMPLE 11

An ointment according to the present invention was prepared from the following ingredients:

| | % by Weight | Amount |
|---|---|---|
| 1-Isobutyl-1H-imidazo[4,5-c]- | 1.0 | 0.20 g |
| quinolin-4-amine | | |
| Isostearic acid | 5.0 | 1.00 g |
| Mineral oil | 12.8 | 2.56 g |
| White petrolatum | 65.2 | 13.04 g |
| Cetyl alcohol | 4.0 | 0.80 g |
| Acetylated lanolin | 1.0 | 0.20 g |
| Witconol .TM. | 143.0 | 0.60 g |
| Aluminum stearate | 8.0 | 1.60 g |

The materials listed above were combined according to following procedure:

The 1-isobutyl-1H-imidazo[4,5-c]quinolin-4-amine and the isostearic acid were placed in a glass jar and heated with stirring until the amine was dissolved. The remaining ingredients were added and the resulting mixture was heated to 65° C. and then mixed while being allowed to cool to room temperature.

### EXAMPLE 12

Using the general procedure of Example 11 an ointment containing the following ingredients was prepared.

| | % by Weight | Amount |
|---|---|---|
| 1-Isobutyl-1H-imidazo[4,5-c]- | 1.0 | 0.20 g |
| quinolin-4-amine | | |
| Isostearic acid | 6.0 | 1.20 g |
| Polyethylene Glycol 400 | 55.8 | 11.16 g |
| Polyethylene Glycol 3350 | 32.6 | 6.52 g |
| Stearyl alcohol | 4.6 | 0.92 g |

### EXAMPLES 13-15

Creams of the present invention were prepared using the ingredients shown in Table 3. The Example 1 except that benzyl alcohol was used with the isostearic acid to dissolve the 1-isobutyl-1H-imidazo[4,5-c]quinolin-4-amine.

TABLE 3

| | Example | | |
|---|---|---|---|
| | 13 | 14 | 15 |
| | % by Weight | | |
| Oil Phase | | | |
| 1-Isobutyl-1H-imidazo[4,5-c]- | 5.0 | 5.0 | 4.85 |
| quinolin-4-amine | | | |
| Isostearic acid | 25.0 | 25.0 | 24.3 |
| Benzyl alcohol | 2.0 | 2.0 | 1.94 |
| Cetyl alcohol | 2.2 | 2.2 | 1.16 |
| Stearyl alcohol | 3.1 | 3.1 | 1.75 |
| Petrolatum | 3.0 | — | 2.91 |
| Polysorbate 60 | 3.4 | 3.4 | 4.13 |
| Sorbitan monostearate | 0.6 | 0.6 | 0.73 |
| Stearic acid | — | — | 9.71 |
| Aqueous Phase | | | |
| Glycerin | 2.0 | 2.0 | 1.94 |
| Methylparaben | 0.2 | 0.2 | 0.19 |
| Propylparaben | 0.02 | 0.02 | 0.02 |
| Purified water | 53.48 | 56.48 | 46.39 |

### EXAMPLE 16

A cream according to the present invention was prepared from the following ingredients:

| | % by Weight | Amount |
|---|---|---|
| Oil Phase | | |
| 1-Isobutyl-1H-imidazo[4,5-c]- | 4.0 | 0.80 g |
| quinolin-4-amine | | |
| Isostearic acid | 20.0 | 4.00 g |
| Benzyl alcohol | 2.0 | 0.40 g |
| Cetyl alcohol | 2.2 | 0.49 g |
| Stearyl alcohol | 3.1 | 0.62 g |
| Polysorbate 60 | 3.4 | 0.68 g |
| Sorbitan monostearate | 0.6 | 0.12 g |
| Aqueous Phase | | |
| 1-Isobutyl-1H-imidazo[4,5-c]- | 1.0 | 0.2 g |
| quinolin-4-amine | | |
| Glycerin | 2.0 | 0.4 g |
| 85% Lactic acid | 1.0 | 0.22 g |
| Methylparaben | 0.2 | 0.04 g |
| Propylparaben | 0.02 | 0.004 g |
| Purified water | 60.48 | 12.0 g |

The materials listed above were combined according to the following procedure:

The isostearic acid and 0.8 g of 1-isobutyl-1H-imidazo[4,5-c]quinolin-4-amine were combined in a glass jar and heated with stirring until the amine had dissolved. The remaining oil phase ingredients were added to this solution and the mixture was heated to about 70° C. The aqueous phase ingredients were weighed into a separate beaker and heated with stirring until the amine and the parabens had dissolved. With both phases at about 70° C., the water phase was added to the oil phase and mixed with a propeller until the mixture cooled to room temperature.

### EXAMPLE 17

A mixture of 5.9415 g of the 93:7 isooctyl acrylate:acrylamide adhesive copolymer prepared in PREPARATIVE METHOD 2 above, 1.5126 g isostearic acid, 2.0075 g ethyl oleate, 0.3021 g glyceryl monolaurate, 0.2936 1-isobutyl-1H-imidazo[4,5-c]quinolin-4-amine (micronized) and 23.7 g of 90:10 ethyl acetate:methanol was placed in a small glass jar. The jar was placed on a horizontal shaker and shaken at room temperature for about 13 hours. The formulation was coated at a thickness of 20 mils onto a 5 mil Daubert 164Z liner. The laminate was oven dried for 3 minutes at 105° F., for 2 minutes at 185° F., and for 2 minutes at 210° F. The resulting adhesive coating contained 59.1 percent 93:7 isooctyl acrylate:acylamide adhesive copolymer, 15.0 percent isostearic acid, 20.0 percent ethyl oleate, 3.0 percent glyceryl monolaurate and 2.9 percent 1-isobutyl-1H-imidazo[4,5-c]quinolin-4-amine. The material was then laminated with 3 mil low density polyethylene backing and die cut into 2.056 cm.sup.2 patches.

### EXAMPLES 18-20

### Pressure-Sensitive Adhesive Coated Sheet Materials Prepared Using Unmicronized 1-Isobutyl-1H-imidazo[4,5c]quinolin-4-amine

Using the general method of Example 17 the formulations shown below were prepared. 1-Isobutyl-1H-imidazo[4,5-c]quinolin-4-amine that had been ground with a mortar and pestle was used. The adhesive was the 93:7 isooctyl acrylate:acrylamide copolymer prepared in PREPARATIVE METHOD 1 above. The solvent was 90:10 ethyl acetate:methanol. All formulations were mixed at room temperature.

| | Example | | |
|---|---|---|---|
| | 18 | 19 | 20 |
| 1-Isobutyl-1H-imidazo[4,5-c]- | 5.0 | 3.0 | 3.0 |
| quinolin-4-amine Ethyl oleate | 5.1 | 5.0 | 8.0 |
| Isostearic acid | 10.0 | 10.0 | 6.0 |
| Oleic acid | 20.0 | 20.0 | 13.0 |
| Glyceryl monolaurate | 1.5 | 1.5 | 1.5 |
| N,N-dimethyldodecylamine- | 1.0 | 1.1 | 3.0 |
| N-oxide Adhesive | 57.4 | 59.3 | 65.4 |

### EXAMPLE 21

A formulation with the same components in the same proportions as Example 18 was prepared using a different method. The 1-isobutyl-1H-imidazo[4,5-c]-quinolin-4-amine was combined with the oleic and isostearic acids and shaken at 40° C. until there was complete dissolution of the 1-isobutyl-1H-imidazo-[4,5-c]quinolin-4-amine. The remaining ingredients were added and shaken a 40° C. for 72 hours. Patches measuring 2.056 cm.sup.2 were prepared by the general method of Example 17.

### EXAMPLE 22

A mixture of 2.4734 g 1-isobutyl-1H-imidazo[4,5-c]-quinolin-4-amine 3.3315 g isostearic acid and 6.6763 g oleic acid was prepared. To 1.8738 g of the above mixture was added 2.8750 g of the 93:7 isooctyl acrylate:acryamide adhesive copolymer prepared in PREPARATIVE METHOD 2 above, 0.2548 g of ethyl oleate, 0.0510 g N,N-dimethyl-dodecylamine-N-oxide, 0.0820 g glyceryl monolaurate (from Lauricidin, Inc.) and 14.0457 g of 90:10 ethyl acetate/methanol. The above was shaken for 30 hours at room temperature on a horizontal shaker. Transdermal patches were then prepared generally according to the procedures of Example 17.

### EXAMPLE 23

Aldara® Cream, commercially available via prescription and manufactured by 3M Health Care Limited, Loughborough LE11 1EP England, and distributed by Graceway Pharmaceuticals, LLC, Bristol, Tenn. 37620, is evaluated in two randomized, vehicle-controlled, double-blind trials involving 702 pediatric subjects with molluscum contagiosum (MC) (470 are exposed to Aldara; median age 5 years, range 2-12 years). Subjects apply Aldara® Cream or vehicle 3 times weekly for up to 16 weeks. Complete clearance (no MC lesions) is assessed at Week 18. In Study 1, the complete clearance rate is 24% (52/217) in the Aidara® Cream group compared with 26% (28/106) in the vehicle group. In Study 2, the clearance rates are 24% (60/253) in the Aldara® Cream group as compared with 28% (35/126) in the vehicle group. While these studies may have failed to demonstrate FDA approval efficacy as compared to vehicle, it is believed that in some cases, imiquimod therapy is effective against molluscum contagiosum in children. Similar to the studies conducted in adults, the most frequently reported adverse reaction from 2 studies in children with molluscum contagiosum is application site reaction. Adverse events which occur more frequently in Aldara®-treated subjects as compared with vehicle-treated subjects generally resemble those seen in studies in indications approved for adults and also included otitis media (5% Aldara vs. 3% vehicle) and conjunctivitis (3% Aldara® vs. 2% vehicle). Erythema is the most frequently reported local skin reaction. The severe local skin reactions that are reported by Aidara®-treated subjects in the pediatric studies include erythema (28%), edema (8%), scabbing/crusting (5%), flaking/scaling (5%), erosion (2%) and weeping/exudate (2%).

More particularly, the objective is to evaluate the efficacy of imiquimod cream, 5% (imiquimod) for the treatment of molluscum contagiosum (MC) lesions in pediatric subjects when the cream was applied 3 times per week (3×/wk) for up to 16 weeks. The efficacy of imiquimod is evaluated by assessing MC lesion clearance. The primary efficacy parameter is complete clearance of all MC lesions (complete clinical resolution) from treatment initiation to the week 18/efficacy visit. Secondary efficacy parameters include partial clearance, defined as ≧50% reduction from the baseline lesion count, and the change in total lesion count. Time to complete clearance is also compared between treatment groups. The secondary objective is to evaluate the safety of imiquimod. Safety assessments are conducted throughout the study. Safety was assessed through incidence and severity of adverse events (AEs); local skin reactions (LSRs) of erythema, edema, erosion/ulceration, weeping/exudates, scabbing/crusting, and flaking/scaling/dryness; skin quality assessments; vital signs measurements; general physical examinations; hematology laboratory tests; and concomitant medication use.

This is a randomized, vehicle-controlled, double-blind, parallel group, study that is conducted in children aged 2 to 12 years of age who had ≧2 clinically verified MC lesions. Enrollment is monitored to ensure at least 50% of subjects had at least 5 MC lesions, at least 50% of subjects are under 6 years of age, and a sufficient number of subjects enrolled with periocular lesions. Subjects are randomized 2:1 (imiquimod: vehicle) and apply study cream 3×/wk for up to 16 weeks (or until complete resolution of all MC lesions) to all target MC lesions. Subjects who weigh <25 kg apply up to 2 sachets of study cream, while subjects who weigh ≧25 kg apply up to 3 sachets. At the screening/treatment initiation visit, MC lesions and application area locations are recorded on a body diagram. Subjects report to the clinic at treatment weeks 2, 4, 8, 12, and 16, and post treatment at week 18 for efficacy assessments, and week 28 for end-of-study procedures. At each study visit, MC lesions are counted and recorded, and safety procedures are performed. Also, the subject's diary is reviewed for compliance. Subjects return at week 18 for the primary efficacy and post treatment safety assessments. All subjects who did not clear their MC lesions as well as subjects who did not complete the 16 weeks of treatment are to report to the clinic at week 28 for final safety and efficacy assessments. Eligible subjects have ≧2 clinically verified MC lesions (at least half had ≧5 lesions) not located on buttocks, or inguinal region or on hands only and are 2 to 12 years old. Imiquimod cream, 5%, 1 to 3 sachets applied to MC lesions, topical. MC lesions are treated 3×/wk for up to 16 weeks, or until determined that all MC lesions are cleared. The vehicle cream, 1 to 3 packets, is applied to MC lesions, topical.

Efficacy is monitored at each study visit by counting MC lesions.

Safety is assessed throughout the study by monitoring AEs, LSRs (erythema, edema, erosion/ulceration, weeping/exudate, flaking/scaling/dryness, and cabbing/crusting), vital signs measurements, and concomitant medication use at all study visits. At specified study visits, skin quality assessments, physical examinations, and photographs of application areas are done. Hematology tests are performed on approximately 25% of subjects.

The primary dataset is analyzed for efficacy and safety is the intent-to-treat (ITT) dataset, consisting of all randomized subjects. A per-protocol (PP) dataset is also analyzed for efficacy. The PP dataset includes data from subjects who have an assessment of the primary variable, apply at least two thirds of the required doses, and are free from major protocol violations. Treatment groups are compared with respect to the primary variable (complete clearance rate at the week 18 study visit) by means of the Cochran-Mantel-Haenzsel (CMH) Test, which adjusts for multiple study centers. The CMH Test is also performed for the secondary efficacy variable: partial clearance rate.

The generalized Wilcoxon Test is used to compare the time (number of study weeks) to complete clearance of MC lesions between treatment groups. All statistical tests are 2-sided and conducted at the alpha=0.05 level. All safety data are tabulated separately by treatment group using the ITT dataset.

Demographics is N=323 and the populations is as follows: Females n=160 (49.5%) Males n=163 (50.5%). The mean (±SD)=5.2±2.40 Range 2 to 12. The race is as follows: White n=305 (94.4%) Black n=15 (4.6%), American Indian/Alaskan n=0 (0%), Asian n=1 (0.3%), Native Hawaiian/Other Pacific Islander n=2 (0.6%). Hispanic/Latino n=42 (13.0%) Non-Hispanic/Latino n=280 (86.7%) Unknown n=1 (0.3%).

For the ITT dataset, subjects in the imiquimod group has a complete clearance rate at week 18 of 24.0% (521217) versus (vs) 26.4% (28/106) for the vehicle group (p=0.6638). In this study, the imiquimod 3×/wk for up to 16 weeks is not significantly different than vehicle with respect to complete clearance of MC lesions at the week 18/efficacy assessment visit. Subjects in the imiquimod group had a partial clearance rate at week 18 of 49.8% (108/217) vs 45.3% (48/106) for the vehicle group (p=0.4416); partial clearance rates between treatment groups were not significantly different. The median percent reduction in MC lesions at week 18 was 48.1% for the imiquimod group and 37.3% for the vehicle group. The distribution in time to complete clearance is not significantly different (p=0.7830) between the imiquimod and vehicle groups. For the imiquimod and vehicle groups, the Cochran-Armitage Test for trend does not identify any statistically significant associations between LSR intensity and complete clearance. While this study indicates that imiquimod is not statistically different than vehicle in treating molluscum contagiosum, there nevertheless are instances when the use of imiquimod is believed to be effective against molluscum contagiosum.

The median cumulative exposure for imiquimod subjects during the study is 587.5 mg imiquimod (range, 12.5 to 1662.5 mg). The median number of doses received is 44.0 (range, 1 to 64). Adverse events occur in both the imiquimod and vehicle groups. The most frequently reported AE is application site reaction, with erythema at target site, itching at target site, and irritation at target site is reported most often. At least 1 AE is considered possibly or probably related to study drug is reported by 33.6% of imiquimod subjects and 19.8% of vehicle subjects (p=0.013). Three subjects (2 imiquimod, 1 vehicle) are discontinued from the treatment period due to AEs, none of which are severe in intensity. Three subjects reported 5 serious adverse events (SAEs). No SAEs are considered related to study drug. No deaths occurred during the study. Of the LSRs assessed by the investigator, there are statistically significant treatment differences in the distribution of maximum severity scores between the imiquimod and vehicle groups for all 6 LSR categories: erythema, edema, erosion/ulceration, weeping/exudate, flaking/scaling/dryness, and scabbing/crusting. Erythema was the most frequently recorded LSR. No subjects are discontinued during the treatment period due to LSRs. There are no significant differences between treatment groups in the distribution of maximum severity scores for 4 skin quality measurements: hyperpigmentation, hypopigmentation, scarring, and atrophy. Findings from the physical examination, vitals signs, and laboratory assessments are consistent with the age of this subject population.

Systemic absorption of imiquimod across the affected skin of 22 subjects aged 2 to 12 years with extensive MC involving at least 10% of the total body surface area is observed after single and multiple doses at a dosing frequency of 3 applications per week for 4 weeks. The investigator determines the dose applied, i.e., either 1, 2 or 3 packets per dose, based on the size of the treatment area and the subject's weight. The overall median peak serum drug concentrations at the end of week 4 is between 0.26 and 1.06 ng/ml except in a 2-year old female who is administered 2 packets of study drug per dose, has a Cmax of 9.66 ng/ml after multiple dosing. Children aged 2-5 years receive doses of 12.5 mg (one packet) or 25 mg (two packets) of imiquimod and have median multiple-dose peak serum drug levels of approximately 0.2 or 0.5 ng/ml, respectively. Children aged 6-12 years receive doses of 12.5 mg, 25 mg, or 37.5 mg (three packets) and have median multiple dose serum drug levels of approximately 0.1, 0.15, or 0.3 ng/ml, respectively. Among the 20 subjects with evaluable laboratory assessments, the median WBC count decreases by 1.4*10⁹/L and the median absolute neutrophil count decreases by 1.42*10⁹/L.

In this study, imiquimod is found to be safe in children ages 2 to 12 with MC when dosed 3×/wk for up to 16 weeks. According to this study, imiquimod is not statistically significantly more effective than vehicle cream when dosed 3×/wk for up to 16 weeks with respect to complete and partial clearance of MC lesions. While this study appears to demonstrate that imiquimod use is safe in children, it is believed that systemic absorption should be monitored during therapy and if serum imiquimod concentrations exceed more than about 2 ng/mL, or there is a decrease in median white blood cell counts by at least about 1.4*10⁹/L or there is a decrease in median absolute neutrophil counts by at least about 1.42*10⁹/L, it is believed that consideration should be given to tapering or stopping treatment, or reducing the dose or frequency of administration, until the symptoms have subsided as precautionary and safety measures.

Aldara® (imiquimod) Cream, 5%, is supplied in single-use packets which contain 250 mg of the cream. It is commercially available to patients by prescription as a box of 12 packets NDC 29336-610-12. It is recommended to store Aldara® Cream at 4-25° C. (39-77° F.) and avoid freezing. Aldara® (imiquimod 5%) Cream is an immune response modifier for topical administration. Each gram contains 50 mg of imiquimod in an off-white oil-in-water vanishing cream base consisting of isostearic acid, cetyl alcohol, stearyl alcohol, white petrolatum, polysorbate 60, sorbitan monostearate, glycerin, xanthan gum, purified water, benzyl alcohol, methylparaben, and propylparaben. The FDA approved-label(s) or package insert(s) for Aldara® Cream are incorporated herein by reference in their entireties. Chemically, imiquimod is 1-(2-methylpropyl)-1H-imidazo[4,5-c]quinolin-4-amine. Imiquimod has a molecular formula of C₁₄H₁₆N₄ and a molecular weight of 240.3.

What is claimed is:

1. A method of using imiquimod safely to treat a child having a skin disorder comprising:
providing information that systemic absorption of imiquimod may be observed after application of imiquimod to skin of the child affected with the skin disorder.

2. The method of claim 1, wherein said method includes the further step of providing information that systemic absorption of imiquimod may be observed after application of a single dose or multiple doses of imiquimod to skin of the child affected with the skin disorder at a dosing frequency of 3 applications per week for 4 weeks.

3. The method of claim 1, wherein said method includes the further step of providing information that the child is of an age between about 2 and about 12 years old.

4. The method of claim 1 or 2, wherein said method further includes the step of providing information that medical evaluation may be required if such systemic absorption is experienced by the child treated with imiquimod.

5. The method of any one of claims 1, 2, 3 or 4, wherein said method further includes the step of providing information that the skin disorder is selected from the group consisting of genital warts, actinic keratosis, superficial basal cell carcinoma and molluscum contagiosum.

6. The method of any one of claims 1, 2, 3 or 4, wherein said method further includes the step of providing information that the skin disorder is molluscum contagiosum.

7. The method of any one of claims 1, 2, 3 or 4, wherein said method further includes the step of providing information that the skin disorder is selected from the group consisting of external genital warts, perianal warts and condyloma acuminata, in a child 12 years or older.

8. The method of any one of claims 1, 2, 3, 4, 5, 6 or 7, wherein said method further includes the step of providing information that the imiquimod is provided as an imiquimod 5% cream for topical use.

9. The method of any one of claims 1, 2, 3, 4, 5, 6, 7 or 8, wherein the information is provided in a package drug insert.

10. The method of any one of claims 1, 2, 3, 4, 5, 6, 7 or 8, wherein the information is provided in a label for imiquimod approved by the FDA.

11. A method of using imiquimod safely as therapy to treat a skin disorder in a child of an age between about 2 and about 12 comprising:
(a) providing the child, or a guardian of the child, with a therapeutically effective amount of imiquimod for application to an area of the child's skin affected with the skin disorder; and
(b) providing information that systemic absorption of imiquimod across the child's skin may be observed.

12. The method of claim 11, wherein said method further includes the step of providing information that systemic absorption of imiquimod across the child's skin may be observed after application of a single dose or multiple doses of imiquimod to the skin disorder.

13. The method of claim 11, wherein said method further includes the step of providing information that systemic absorption of imiquimod across the child's skin may be observed after application of a single dose or multiple doses of imiquimod to the skin disorder at a dosing frequency of 3 applications per week for 4 weeks.

14. The method of any of claims 11-13, wherein said method further includes the step of providing information that medical evaluation may be required if such systemic absorption is experienced by the child.

15. The method of any one of claims 11-13, wherein said method further includes the step of providing information that the skin disorder is selected from the group consisting of genital warts, perianal warts, condyloma acuminata, actinic keratosis, superficial basal cell carcinoma and molluscum contagiosum.

16. The method of any one of claims 11-13, wherein said method further includes the step of providing information that the skin disorder is molluscum contagiosum.

17. The method of any one of claims 11-13, wherein the skin disorder is selected from the group consisting of genital warts, perianal warts and condyloma acuminata.

18. The method of any one of claims 11-13, wherein said method further includes the step of providing information that the skin disorder is actinic keratosis.

19. The method of any one of claims 11-13, wherein said method further includes the step of providing information that the skin disorder is superficial basal cell carcinoma.

20. The method of any one of claims 11-19, wherein said method further includes the step of providing information that the imiquimod is provided at a dose of between about 12.5 mg and 37.5 mg.

21. The method of any one of claims 11-19, wherein said method further includes the step of providing information that the imiquimod is provided at a dose selected from the group consisting of about 12.5 mg, about 25 mg and about 37.5 mg.

22. The method of any one of claims 11-21, wherein said method further includes the step of providing information that the imiquimod is provided in unit dose form.

23. The method of any one of claims 11-21, wherein said method further includes the step of providing information that the imiquimod is provided in a unit dose or in multiple doses to provide for a course of therapy.

24. The method of any one of claims 22 or 23, wherein said method further includes the step of providing information that the imiquimod unit dose is in an amount of about 12.5 mg.

25. The method of claim 23, wherein said method further includes the step of providing information that the imiquimod multiple doses are in an amount selected from the group consisting of about 25 my and about 37.5 mg.

26. The method of any one of claims 11-25, said method further includes the step of providing information to inform, the child, a guardian of the child or a physician treating the child that peak serum imiquimod concentration achieved during the therapy following either single or multiple doses therapy is less than about 2 ng/ml.

27. The method of any one of claims 11-25, wherein said method further includes the step of providing information to inform the child, any one of claims 1-13 guardian of the child or a physician treating the child's that if the child, whose age is between about 2 and about 5 years old, receives doses of about 12.5 mg or about 25 mg of imiquimod, multiple dose-peak serum imiquimod levels of about 0.2 ng/ml or about 0.5 ng/ml, respectively, may be achieved.

28. The method of any one of claims 11-25, said method further includes the step of providing information to inform the child, a guardian of the child or a physician treating the child that if the child, whose age is between about 6 and about 12 years old, receives doses of about 12.5 mg, about 25 mg or 37.5 mg of imiquimod, median multiple dose serum imiquimod levels of about 0.1 ng/ml, about 0.15 ng/ml or 0.3 ng/ml, respectively, may be achieved.

29. The method of any one of claims 11-28, wherein said method further includes the step of providing information that the imiquimod is provided as an imiquimod 5% cream for topical use.

30. The method of any one of claims 11-29, wherein the information is provided in a package drug insert.

31. The method of any one of claims 11-29, wherein the information is provided in a label for imiquimod approved by the FDA.

32. A safer method of using imiquimod as a therapy to treat a topical skin disorder in a child between about 2 and about 12 years of age comprising:
(a) providing the child, a guardian of the child or a physician treating the child with a therapeutically effective amount of imiquimod for topical application to an area of the child's skin affected with the skin disorder; and
(b) informing the child, the guardian or the treating physician that a decrease in the child's median white blood cell count by about 1.4*109/L or a decrease in the child's median absolute neutrophil count of about 1.42*109/L may be observed during the imiquimod therapy.

33. The method of claim 32, wherein said method includes the further step of advising the child, the child's guardian or the child's treating physician that prompt medical evaluation may be required if the decrease in the median white blood cell count or the decrease in the median absolute neutrophil count is experienced by the child.

34. The method of any one of claims 32 or 33, wherein said method further includes the step of providing information that the skin disorder is selected from the group consisting of genital warts, perianal warts, condyloma acuminata, actinic keratosis, superficial basal cell carcinoma and molluscum contagiosum.

35. The method of any one of claims 32 or 33, wherein said method further includes the step of providing information that the skin disorder is molluscum contagiosum.

36. The method of any one of claims 32 or 33, wherein the skin disorder is selected from the group consisting of genital warts, perianal warts and condyloma acuminata.

37. The method of any one of claims 32 or 33, wherein said method further includes the step of providing information that the skin disorder is actinic keratosis.

38. The method of any one of claims 32 or 33 wherein said method further includes the step of providing information that the skin disorder is superficial basal cell carcinoma.

39. The method of any one of claims 32-37, wherein said method further includes the step of providing information that the imiquimod is provided at a dose of between about 12.5 mg and 37.5 mg.

40. The method of any one of claims 32-37, wherein said method further includes the step of providing information that the imiquimod is provided at a dose selected from the group consisting of about 12.5 mg, about 25 mg and about 37.5 mg.

41. The method of any one of claims 32-40, wherein said method further includes the step of providing information that the imiquimod is provided in unit dose form.

42. The method of any one of claims 32-40, wherein said method further includes the step of providing information that the imiquimod is provided in a unit dose or in multiple doses to provide for a course of therapy.

43. The method of any one of claims 32-40, wherein said method further includes the step of providing information that the imiquimod unit dose is in an of about 12.5 mg.

44. The method of claim 43, wherein said method further includes the step of providing information that the imiquimod multiple doses are in an amount selected from the group consisting of about 25 mg and about 37.5 mg.

45. The method of any one of claims 32-43, wherein said method further includes the step of providing information to inform, the child, a guardian of the child or a physician treating the child that peak serum imiquimod concentration achieved during the therapy following either single or multiple doses therapy is less than about 2 ng/ml.

46. The method of any one of claims 32-43, wherein said method further includes the step of providing information to inform the child, a guardian of the child or a physician treating the child's that if the child, whose age is between about 2 and about 5 years old, receives doses of about 12.5 mg or about 25 mg of imiquimod, multiple dose-peak serum imiquimod levels of about 0.2 ng/ml or about 0.5 ng/ml, respectively, may be achieved.

47. The method of any one of claims 32-43, wherein said method further includes the step of providing information to inform the child, a guardian of the child or a physician treating the child that if the child, whose age is between about 6 and about 12 years old, receives doses of about 12.5 my, about 25 mg or 37.5 mg of imiquimod, median multiple dose serum imiquimod levels of about 0.1 ng/ml, about 0.15 ng/ml or 0.3 ng/ml, respectively, may be achieved.

48. The method of any one of claims 32-47, wherein said method further includes the step of providing information that the imiquimod is provided as an imiquimod 5% cream for topical use.

49. The method of any one of claims 32-48, wherein the information is provided in a package drug insert.

50. The method of any one of claims 32-48, wherein the information is provided in a label for imiquimod approved by the FDA.

51. A method of using imiquimod as therapy for treating a child diagnosed skin disorder comprising:
enhancing the safety profile of imiquimod by (i) providing information to a prescribing physician that systemic absorption of imiquimod may result in the child receiving imiquimod therapy, and (ii) informing the prescribing physician to monitor the child who is prescribed imiquimod for treating the child's skin disorder.

52. The method of claim 51, wherein said method includes the further step of providing information that the child is between about 2 and about 12 years of age.

53. The method of any one of claims 51 or 52, wherein said method further includes providing information to the physician that the monitoring comprises measuring serum imiquimod levels in the child during the imiquimod therapy.

54. The method of any one of claims 51-53, wherein said method further includes providing information to inform the physician that the monitoring comprises measuring for decreases in normal median white blood cell counts or median absolute neutrophil counts.

55. The method of any one of claims 51-54, wherein said method further includes the step of providing information to inform the physician that peak serum imiquimod concentration achieved in the child during the therapy following either single or multiple doses therapy is less than about 2 ng/ml.

56. The method of any one of claims 51-54, wherein said method further includes the step of providing information to inform the physician that if the child, whose age is between about 2 and about 5 years old, receives doses of about 12.5 mg or about 25 mg of imiquimod, multiple dose-peak serum imiquimod levels of about 0.2 ng/ml or about 0.5 ng/ml, respectively, may be achieved.

57. The method of any one of claims 51-54, wherein said method further includes the step of providing information to inform the physician that if the child, whose age is between about 6 and about 12 years old, receives doses of about 12.5 mg, about 25 mg or 37.5 mg of imiquimod, median multiple dose serum imiquimod levels of about 0.1 ng/ml, about 0.15 ng/ml or 0.3 ng/ml, respectively, may be achieved.

58. The method of any one of claims 51-57, wherein said method includes providing information to the physician that when systemic absorption of imiquimod is observed, the physician should stop, reduce or taper-off imiquimod dosing in the child.

59. The method of any one of claims 51-57, wherein said method further includes providing information to the physician that once serum levels in the child with respect to imiquimod return to normal or near normal, the physician may restart imiquimod therapy.

60. The method of any one of claims 51-59, wherein said method further includes the step of providing information that the imiquimod is provided as an imiquimod 5% cream for topical use.

61. The method of any one of claims 51-60, wherein the information is provided in a package drug insert.

62. The method of any one of claims 51-60, wherein the information is provided in a label for imiquimod approved by the FDA.

63. A method of using imiquimod as topical therapy for treating a child diagnosed with a skin disorder comprising:
(a) providing packaging that includes a topical pharmaceutical formulation of imiquimod; and
(b) providing information with or separate from the packaging that the topical pharmaceutical formulation during the topical imiquimod therapy may cause systemic absorption of imiquimod in the child.

64. A method of claim 63, wherein said method includes the further step of providing the packaging to the child, or a guardian of the child, who has been prescribed imiquimod to treat the child's skin disorder.

65. A method of any one of claims 63-64, wherein said method further includes the further step of providing the information to a nurse or physician treating the child who has been prescribed imiquimod to treat the child's skin disorder.

66. A method of any one of claims 63-65, wherein said method includes the further step of providing information that one or more symptoms selected from the group consisting of a decrease in the child's median white blood cell count or a decrease in the child's median absolute neutrophil count are potential signs of systemic absorption of imiquimod into the blood or serum of the child during the topical imiquimod therapy.

67. A method of any one of claims 63-65, wherein said method includes the further step of providing information to the child or a guardian of the child that a decrease in the child's median white blood cell count is a potential sign of systemic absorption of imiquimod.

68. A method of any one of claims 63-65, wherein said method includes the further step of providing information to a nurse or physician treating the child that a decrease in the child's median white blood cell count is a potential sign of systemic absorption of imiquimod.

69. A method of any one of claims 63-65, wherein said method includes the further step of providing information to the child or a guardian of the child that a decrease in the child's median absolute neutrophil count is a potential sign of systemic absorption of imiquimod.

70. A method of any one of claims 63-65, wherein said method includes the further step of providing information to a nurse or physician treat the child that a decrease in the child's median absolute neutrophil count is a potential sign of systemic absorption of imiquimod.

71. A method of any one of claims 63-65, wherein said method includes the further step of providing information to the child, a guardian of the child or a nurse or physician treating the child that one or more symptoms selected from the group consisting of a decrease in the child's median white blood cell count and a decrease in the child's median absolute neutrophil count is a sign of systemic absorption of imiquimod.

72. A method of any of the claims of 63-65, wherein said method includes the further step of providing information to the child, a guardian of the child or a nurse or physician treating the child that a decrease in the child's median white blood cell count is a sign of systemic absorption of imiquimod.

73. A method of any of the claims of 63-65, wherein said method includes the further step of providing information to the child, a guardian of the child or a nurse or physician treating the child, or a guardian of the child, that a decrease in the child's median absolute neutrophil count is a sign of systemic absorption of imiquimod.

74. A method of any of the claims of 63-65, wherein said method includes the further step of providing information to the child, a guardian of the child or a nurse or physician treating the child that one or more symptoms selected from the group consisting of a decrease in the child's median white blood cell count and a decrease in the child's median absolute neutrophil count are side effects of using imiquimod.

75. A method of any of the claims of 63-65, wherein said method includes the further step of providing information to the child, a guardian of the child or a nurse or physician treating the child that a decrease in the child's median white blood cell count is a side effect of using imiquimod.

76. A method of any of the claims of 63-65, wherein said method includes the further step of providing information to the child, a guardian of the child or a nurse or physician treating the child that a decrease in the child's median absolute neutrophil count is a side effect of using imiquimod.

77. A method of any of the claims of 63-76, wherein said method includes the further step of providing information to the child, a guardian of the child or a nurse or physician treating the child that a median multiple-dose peak serum imiquimod level of less than about 0.2 ng/ml is a side effect of using the imiquimod.

78. A method of any of the claims of 63-76, wherein said method includes the further step of providing information to the child, a guardian of the child or a nurse or physician treating the child that a median multiple-dose peak serum imiquimod level of about 0.2 or about 0.5 ng/ml is a side effect of using the imiquimod.

79. A method of any of the claims of 1-14, wherein said method includes the further step of providing information to the child, a guardian of the child or a nurse or physician treating the child that a median multiple-dose peak serum imiquimod level, selected from the group of about 0.1, about 0.15 ng/ml and about 0.3 ng/ml, is a side effect of using the imiquimod.

80. The method of any one of claims 63-76, wherein said method includes the further step of providing information that the skin disorder is molluscum contagiosum.

81. The method of any one of claims 63-76, wherein said method includes the further step of providing information that the skin disorder is selected from the group consisting of genital warts, perianal warts and condyloma acuminata.

82. The method of any one of claims 63-76, wherein said method includes the further step of providing information that the skin disorder is actinic keratosis.

83. The method of any one of claims 63-76, wherein said method includes the further step of providing information that the skin disorder is superficial basal cell carcinoma.

84. The method of any one of claims 63-83, wherein said method includes the further step of providing information that the child is between about 2 and about 12 years of age.

85. The method of any one of claims 63-83, wherein said method includes the further step of providing information that the child is between about 2 and about 5 years of age.

86. The method of any one of claims 63-83 wherein said method further includes the step of providing information that the imiquimod is provided as an imiquimod 5% cream for topical use.

87. The method of any one of claims 63-84, wherein the information is provided in a package drug insert.

88. The method of any one of claims 63-84, wherein the information is provided in a label for imiquimod approved by the FDA.

89. A method of using imiquimod as topical therapy for treating a skin disorder comprising:
(a) providing packaging that includes a topical pharmaceutical formulation of imiquimod; and
(b) providing information that the topical pharmaceutical formulation may cause in a child, between about 2 and about 12 years of age, a peak serum imiquimod concentration following a single dose or multiple doses of less than 2 ng/ml or less than about 2 ng/ml.

90. A method of claim 89, wherein said method includes the further step of providing the packaging to the child who has been prescribed imiquimod to treat the skin disorder.

91. A method of any one of claims 89-90, wherein said method includes the further step of providing the information to a guardian of the child or a nurse or physician treating the child who has been prescribed imiquimod to treat the skin disorder.

92. A method of any one of claims 89-91, wherein said method includes the further step of providing information that one or more symptoms selected from the group consisting of a decrease in the child's median white blood cell count and a decrease in the child's median absolute neutrophil count are potential signs of systemic absorption of imiquimod.

93. A method of any one of claims 89-92, wherein said method includes the further step of providing information that a decrease in the child's median white blood cell count is a potential sign of systemic absorption of imiquimod.

94. A method of any one of claims 89-92, wherein said method includes the further step of providing information that a decrease in the child's median absolute neutrophil count is a potential sign of systemic absorption of imiquimod.

95. A method of any one of claims 89-94, wherein said method includes the further step of providing information that the skin disorder is molluscum contagiosum.

96. A method of any one of claims 89-94, wherein said method includes the further step of providing information that the skin disorder is selected from the group consisting of genital warts, perianal warts and condyloma acuminata.

97. A method of any one of claims 89-94, wherein said method includes the further step of providing information that the skin disorder is actinic keratosis.

98. The method of any one of claims 89-94, wherein said method includes the further step of providing information that the skin disorder is superficial basal cell carcinoma.

99. The method of any one of claims 89-98, wherein said method includes the further step of providing information that a child between about 6 and about 12 years of age received topical imiquimod doses selected from the group consisting of about 12.5 mg, about 25 mg and about 37.5 mg.

100. The method of any one of claims 89-99, wherein said method includes the further step of providing information that a child between about 6 and about 12 years of age received topical imiquimod doses selected from the group consisting of about 12.5 mg, about 25 mg and about 37.5 mg and had median multiple dose serum drug levels of approximately 0.1, 0.15, or 0.3 ng/mL, respectively.

101. The method of any one of claims 89-100, wherein said method further includes the step of providing information that the imiquimod is provided as an imiquimod 5% cream for topical use.

102. The method of any one of claims 89-101, wherein the information is provided in a package drug insert.

103. The method of any one of claims 89-101, wherein the information is provided in a label for imiquimod approved by the FDA.

104. A method of using imiquimod as topical therapy for treating a skin disorder comprising:
(a) providing packaging that includes a topical pharmaceutical formulation of imiquimod; and
(b) providing information that the topical pharmaceutical formulation may cause in a child, between about 2 and about 5 years of age, a median multiple-dose peak serum imiquimod level of about 0.2 or about 0.5 ng/ml.

105. The method of claim 104, wherein said method includes the further step of providing the packaging to the child who has been prescribed imiquimod to treat the skin disorder, or a guardian of the child.

106. The method of any one of claims 104 or 105, wherein said method includes the further step of providing the information to the child who has been prescribed imiquimod to treat the skin disorder, or a guardian for the child or a nurse or a physician treating the child.

107. The method of any one of claims 104-106, wherein said method includes the further step of providing information that one or more symptoms selected from the group consisting of a decrease in the child's median white blood cell count and a decrease in the child's median absolute neutrophil count are potential signs of systemic absorption of imiquimod.

108. The method of any one of claims 104-107, wherein said method includes the further step of providing information that a median multiple-dose peak serum imiquimod level of about 0.2 or about 0.5 ng/ml is a sign of systemic absorption of imiquimod.

109. The method of any one of claims 104-108, wherein said method includes the further step of providing information that a decrease in the child's median white blood cell count and a decrease in the child's median absolute neutrophil count are potential signs of systemic absorption of imiquimod.

110. The method of any one of claims 104-109, wherein said method includes the further step of providing information that a decrease in the child's median white blood cell count is a sign of systemic absorption of imiquimod.

111. The method of any one of claims 104-110, wherein said method includes the further step of providing information that a decrease in the child's median absolute neutrophil count is a sign of systemic absorption of imiquimod.

112. The method of any one of claims 104-111, wherein said method includes the further step of providing information that systemic absorption of imiquimod is a side effect of using the imiquimod topical pharmaceutical formulation.

113. The method of any one of claims 104-112, wherein said method includes the further step of providing information that a decrease in the child's median white blood cell count is a side effect of using the imiquimod topical pharmaceutical formulation.

114. The method of any one of claims 104-113, wherein said method includes the further step of providing information that a decrease in the child's median absolute neutrophil count is a side effect of using the imiquimod topical pharmaceutical formulation.

115. The method of any one of claims 104-114, wherein said method includes the further step of providing information that a median multiple-dose peak serum imiquimod level of about 0.2 or about 0.5 ng/ml is a side effect of using the imiquimod topical pharmaceutical formulation.

116. The method of any one of claims 104-115, wherein said method includes the further step of providing information that the skin disorder is molluscum contagiosum.

117. The method of any one of claims 104-116, wherein said method includes the further step of providing information that the skin disorder is selected from a group consisting of genital warts, perianal warts and condyloma acuminata.

118. The method of any one of claims 104-117, wherein said method includes the further step of providing information that the skin disorder is actinic keratosis.

119. The method of any one of claims 104-118, wherein said method includes the further step of providing information that the skin disorder is superficial basal cell carcinoma.

120. The method of any one of claims 104-119, wherein said method includes the further step of providing information that a child between about 6 and about 12 years of age received topical imiquimod doses selected from the group consisting of about 12.5 mg, about 25 mg and about 37.5 mg.

121. The method of claim 120, wherein said method includes the further step of providing information that a child between about 6 and about 12 years of age received topical imiquimod doses selected from the group consisting of about 12.5 mg, about 25 mg and about 37.5 mg and had median multiple dose serum drug levels of approximately 0.1, 0.15, or 0.3 ng/mL, respectively.

122. The method of any one of claims 104-121, wherein said method further includes the step of providing information that the imiquimod is provided as an imiquimod 5% cream for topical use.

123. The method of any one of claims 104-122, wherein the information is provided in a package drug insert.

124. The method of any one of claims 104-122, wherein the information is provided in a label for imiquimod approved by the FDA.

125. A method of using imiquimod as topical therapy for treating a skin disorder comprising:
(a) providing packaging that includes a topical pharmaceutical formulation of imiquimod; and
(b) providing information that the topical pharmaceutical formulation may cause in a child, between about 6 and about 12 years of age, a median multiple-dose peak serum imiquimod level selected from the group of about 0.1, about 0.15 ng/ml and about 0.3 ng/ml.

126. The method of claim 125, wherein said method includes the further step of providing the packaging to the child, or a guardian for the child, who has been prescribed imiquimod to treat the skin disorder.

127. The method of any one of claims 125 or 126, wherein said method includes the further step of providing the information to the child who has been prescribed imiquimod to treat the skin disorder, or a guardian for the child or a nurse or physician treating the child.

128. The method of any one of claims 125-127, wherein said method includes the further step of providing information that one or more symptoms selected from the group consisting of a decrease in the child's median white blood cell count and a decrease in the child's median absolute neutrophil count are potential signs of systemic absorption of imiquimod.

129. The method of any one of claims 125-128, wherein said method includes the further step of providing information that a decrease in the child's median white blood cell count is a sign of systemic absorption of imiquimod.

130. The method of any one of claims 125-129, wherein said method includes the further step of providing information that a decrease in the child's median absolute neutrophil count is a sign of systemic absorption of imiquimod.

131. The method of any one of claims 125-130, wherein said method includes the further step of providing information that a median multiple-dose peak serum imiquimod level selected from a group of about 0.1, about 0.15 ng/ml and about 0.3 ng/ml is a sign of systemic absorption of imiquimod.

132. The method of any one of claims 125-131, wherein said method includes the further step of providing information that systemic absorption of imiquimod is a side effect of using the imiquimod topical pharmaceutical formulation.

133. The method of any one of claims 125-132, wherein said method includes the further step of providing information that a decrease in the child's median white blood cell count is a side effect of using the imiquimod topical pharmaceutical formulation.

134. The method of any one of claims 125-133, wherein said method includes the further step of providing information that a decrease in the child's median absolute neutrophil count is a side effect of using the imiquimod topical pharmaceutical formulation.

135. The method of any one of claims 125-134, wherein said method includes the further step of providing information that a median multiple-dose peak serum imiquimod level selected from the group of about 0.1, about 0.15 ng/ml and about 0.3 ng/ml is a side effect of using the imiquimod topical pharmaceutical formulation.

136. The method of any one of claims 125-135, wherein said method includes the further step of providing information that the skin disorder is molluscum contagiosum.

137. The method of any one of claims 125-136, wherein said method includes the further step of providing information that the skin disorder is selected from a group consisting of genital warts, perianal warts and condyloma acuminata.

138. The method of any one of claims 125-137, wherein said method includes the further step of providing information that the skin disorder is actinic keratosis.

139. The method of any one of claims 125-138, wherein said method includes the further step of providing information that the skin disorder is superficial basal cell carcinoma.

140. The method of any one of claims 125-139, wherein said method includes the further step of providing information that the child is of an age of between about 2 and about 12 years old.

141. The method of any one of claims 125-140, wherein said method further includes the step of providing information that the imiquimod is provided as an imiquimod 5% cream for topical use.

142. The method of any one of claims 125-141, wherein the information is provided in a package drug insert.

143. The method of any one of claims 125-141, wherein the information is provided in a label for imiquimod approved by the FDA.

144. A method of using imiquimod as topical therapy for treating a skin disorder comprising:
(a) providing packaging that includes a topical pharmaceutical formulation of imiquimod; and
(b) providing information that the topical pharmaceutical formulation may cause a decrease in a child's median white blood cell count.

145. The method of claim 144, wherein said method includes the further step providing the packaging to the child, or a guardian for the child, who has been prescribed imiquimod to treat the skin disorder.

146. The method of claim 144, wherein said method includes the further step of providing the information to the child who has been prescribed imiquimod to treat the skin disorder, or a guardian for the child or a nurse or physician treating the child.

147. The method of any one of claims 144-145, wherein said method includes the further step of providing information that a decrease in the child's median white blood cell count is a potential sign of systemic absorption of imiquimod.

148. The method of any one of claims 144-145, wherein said method includes the further step of providing information that a decrease in the child's median white blood cell count is a sign of systemic absorption of imiquimod.

149. The method of any one of claims 144-145, wherein said method includes the further step of providing information that a decrease in the child's median white blood cell count is a side effect of using the imiquimod topical pharmaceutical formulation.

150. The method of any one of claims 144-149, wherein said method includes the further step of providing information that the skin disorder is molluscum contagiosum.

151. The method of any one of claims 144-150, wherein said method includes the further step of providing information that the skin disorder is selected from a group consisting of genital warts, perianal warts and condyloma acuminata.

152. The method of any one of claims 144-151, wherein said method includes the further step of providing information that the skin disorder is actinic keratosis.

153. The method of any one of claims 144-152, wherein said method includes the further step of providing information that the skin disorder is superficial basal cell carcinoma.

154. The method of any one of claims 144-153, wherein said method includes the further step of providing information that the child is of an age of between about 2 and about 12 years old.

155. The method of any one of claims 144-154, wherein said method includes the further step of providing information that the median white blood cell count is decreased by about 1.4*109/L.

156. The method of any one of claims 144-155, wherein said method further includes the step of providing information that the imiquimod is provided as an imiquimod 5% cream for topical use.

157. The method of any one of claims 144-155, wherein the information is provided in a package drug insert.

158. The method of any one of claims 144-155, wherein the information is provided in a label for imiquimod approved by the FDA.

159. A method of using imiquimod as topical therapy for treating a skin disorder comprising:
(a) providing packaging that includes a topical pharmaceutical formulation comprising imiquimod; and
(b) providing information that the topical pharmaceutical formulation may cause a decrease in a child's median absolute neutrophil count.

160. The method of claim 159, wherein said method includes the further step of providing the packaging to the child, or a guardian for the child, who has been prescribed imiquimod to treat the skin disorder.

161. The method of any one of claims 159-160, wherein said method includes the further step of providing the information to the child who has been prescribed imiquimod to treat the skin disorder, or a guardian for the child or a nurse or physician treating the child.

162. The method of any one of claims 159-161, wherein said method includes the further step of providing information that a decrease in the child's median absolute neutrophil count is a potential sign of systemic absorption of imiquimod.

163. The method of any one of claims 159-161, wherein said method includes the further step of providing information that a decrease in the child's median absolute neutrophil count is a sign of systemic absorption of imiquimod.

164. The method of any one of claims 159-161, wherein said method includes the further step of providing information that a decrease in the child's median absolute neutrophil count is a side effect of using the imiquimod topical pharmaceutical formulation.

165. The method of any one of claims 159-164, wherein said method includes the further step of providing information that the skin disorder is molluscum contagiosum.

166. The method of any one of claims 159-165, wherein said method includes the further step of providing information that the skin disorder is selected from a group consisting of genital warts, perianal warts and condyloma acuminata.

167. The method of any one of claims 159-166, wherein said method includes the further step of providing information that the skin disorder is actinic keratosis.

168. The method of any one of claims 159-167, wherein said method includes the further step of providing information that the skin disorder is superficial basal cell carcinoma.

169. The method of any one of claims 159-168, wherein said method includes the further step of providing information that the child is of an age of between about 2 and about 12 years old.

170. The method of any one of claims 159-169, wherein said method includes the further step of providing information that the median absolute neutrophil count decreased by about 1.42*109/L.

171. The method of any one of claims 159-170, wherein said method further includes the step of providing information that the imiquimod is provided as an imiquimod 5% cream for topical use.

172. The method of any one of claims 159-171, wherein the information is provided in a package drug insert.

173. The method of any one of claims 159-171, wherein the information is provided in a label for imiquimod approved by the FDA.

174. A package for enhancing the safety of using imiquimod as topical therapy for treating a skin disorder comprising:
(a) a topical pharmaceutical formulation containing imiquimod for treating the skin disorder; and
(b) information advising that the topical pharmaceutical formulation may cause systemic absorption of imiquimod in a child between the ages of about 2 and about 12.

175. The package of claim 174, wherein said information further includes providing information that systemic absorption is a side effect of using imiquimod to treat the skin disorder.

176. The package of any one of claims 174-175, wherein said information further includes providing information that a decrease in the child's median white blood cell count is a side effect of using imiquimod to treat the skin disorder.

177. The package of any one of claims 174-176, wherein said information further includes providing information that a decrease in the child's median absolute neutrophil count is a side effect of using imiquimod to treat the skin disorder.

178. The package of any one of claims 174-177, wherein said information further includes providing information that a median multiple-dose peak serum imiquimod level of less than about 0.2 ng/ml is a side effect of using imiquimod.

179. The package of any one of claims 174-178, wherein said information further includes providing information that a median multiple-dose peak serum imiquimod level of about 0.2 or about 0.5 ng/ml is a side effect of using the imiquimod.

180. The package of any one of claims 174-179, wherein said information further includes providing information that a median multiple-dose peak serum imiquimod level selected from the group of about 0.1, about 0.15 ng/ml and about 0.3 ng/ml is a side effect of using imiquimod.

181. The package of any one of claims 174-180, wherein said topical pharmaceutical formulation for treating the skin disorder is a 5% imiquimod cream.

182. The package of any one of claims 174-180, wherein said topical pharmaceutical formulation for treating the skin disorder is a 5% imiquimod ointment.

183. The package of any one of claims 174-180, wherein said topical pharmaceutical formulation for treating the skin disorder is a 5% imiquimod salve.

184. The package of any one of claims 174-180, wherein said topical pharmaceutical formulation for treating the skin disorder is a 5% imiquimod lotion.

185. The package of any one of claims 174-180, wherein said topical pharmaceutical formulation for treating the skin disorder is a 5% imiquimod gel.

186. The package of any one of claims 174-180, wherein said topical pharmaceutical formulation for treating the skin disorder is an imiquimod pressure sensitive adhesive coating

187. The package of any one of claims 174-180, wherein said topical pharmaceutical formulation for treating the skin disorder is an imiquimod adhesive-coated sheet material.

188. The package of any one of claims 174-180, wherein said topical pharmaceutical formulation for treating the skin disorder is a 5% imiquimod foam.

189. The package of any one of claims 174-188, wherein the skin disorder is molluscum contagiosum.

190. The package of any one of claims 174-189, wherein the skin disorder is selected from a group consisting of genital warts, perianal warts and condyloma acuminata.

191. The package of any one of claims 174-190, wherein the skin disorder is actinic keratosis.

192. The package of any one of claims 174-191, wherein the skin disorder is superficial basal cell carcinoma.

193. The package of any one of claims 174-192, wherein the information is provided in a package drug insert.

194. The package of any one of claims 174-192, wherein the information is provided in a label for imiquimod approved by the FDA.

195. The package of any one of claims 174-194, wherein the child is between about 2 and about 12 years of age.

196. A package for enhancing the safety of using imiquimod as topical therapy for treating a skin disorder comprising:
(a) a topical pharmaceutical formulation containing imiquimod for treating the skin disorder; and
(b) information advising that the imiquimod topical pharmaceutical formulation may cause a decrease in a child's median white blood cell count.

197. The package of claim 196, wherein said information further includes a warning that a decrease in the child's median white blood cell count is a side effect of using imiquimod to treat the skin disorder.

198. The package of any one of claims 196 or 197, wherein said information further includes information that a median multiple-dose peak serum imiquimod level of less than about 0.2 ng/ml is a side effect of using imiquimod.

199. The package of any one of claims 196-198, wherein said information further includes information that a median multiple-dose peak serum imiquimod level of about 0.2 or about 0.5 ng/ml is a side effect of using the imiquimod.

200. The package of any one of claims 196-199, wherein said information further includes information that a median multiple-dose peak serum imiquimod level selected from the group of about 0.1, about 0.15 ng/ml and about 0.3 ng/ml is a side effect of using imiquimod.

201. The package of any one of claims 196-200, wherein said information further includes information that a decrease in the child's median white blood cell count by about 1.4*10⁹/L is a side effect of using imiquimod.

202. The package of any one of claims 196-201, wherein said topical pharmaceutical formulation for treating the skin disorder is a 5% imiquimod cream.

203. The package of any one of claims 196-201, wherein said topical pharmaceutical formulation for treating the skin disorder is a 5% imiquimod ointment.

204. The package of any one of claims 196-201, wherein said topical pharmaceutical formulation for treating the skin disorder is a 5% imiquimod salve.

205. The package of any one of claims 196-201, wherein said topical pharmaceutical formulation for treating the skin disorder is a 5% imiquimod lotion.

206. The package of any one of claims 196-201, wherein said topical pharmaceutical formulation for treating the skin disorder is a 5% imiquimod gel.

207. The package of any one of claims 196-201, wherein said topical pharmaceutical formulation for treating the skin disorder is an imiquimod pressure sensitive adhesive coating

208. The package of any one of claims 196-201, wherein said topical pharmaceutical formulation for treating the skin disorder is an imiquimod adhesive-coated sheet material.

209. The package of any one of claims 196-201, wherein said topical pharmaceutical formulation for treating the skin disorder is a 5% imiquimod foam.

210. The package of any one of claims 196-209, wherein the skin disorder is molluscum contagiosum.

211. The package of any one of claims 196-210, wherein the skin disorder is selected from a group consisting of genital warts, perianal warts and condyloma acuminata.

212. The package of any one of claims 196-211, wherein the skin disorder is actinic keratosis.

213. The package of any one of claims 196-212, wherein the skin disorder is superficial basal cell carcinoma.

214. The package of any one of claims 196-213, wherein the information is provided in a package drug insert.

215. The package of any one of claims 196-214, wherein the information is provided in a label for imiquimod approved by the FDA.

216. The package of any one of claims 196-215, wherein the child is between about 2 and about 12 years of age.

217. A package for enhancing the safety of using imiquimod as topical therapy for treating a skin disorder comprising:
(a) a topical pharmaceutical formulation containing imiquimod for treating the skin disorder; and
(b) information advising that the imiquimod topical pharmaceutical formulation may cause a decrease in a child's median absolute neutrophil count.

218. The package of claim 217, wherein said information further includes a warning that a decrease in the child's median absolute neutrophil count is a side effect of using imiquimod to treat the skin disorder.

219. The package of any one of claims 217 or 218, wherein said information further includes information that a median multiple-dose peak serum imiquimod level of less than about 0.2 ng/ml is a side effect of using imiquimod.

220. The package of any one of claims 217-219, said wherein information further includes information that a median multiple-dose peak serum imiquimod level of about 0.2 or about 0.5 ng/ml is a side effect of using the imiquimod.

221. The package of any one of claims 217-220, said wherein information further includes information that a median multiple-dose peak serum imiquimod level selected from the group of about 0.1, about 0.15 ng/ml and about 0.3 ng/ml is a side effect of using imiquimod.

222. The package of any one of claims 217-221, said wherein information further includes information that a decrease in the child's median absolute neutrophil count decreased by 1.42*10⁹/L is a side effect of using imiquimod.

223. The package of any one of claims 217-222, wherein said topical pharmaceutical formulation for treating the skin disorder is a 5% imiquimod cream.

224. The package of any one of claims 217-222, wherein said topical pharmaceutical formulation for treating the skin disorder is a 5% imiquimod ointment.

225. The package of any one of claims 217-222, wherein said topical pharmaceutical formulation for treating the skin disorder is a 5% imiquimod salve.

226. The package of any one of claims 217-222, wherein said topical pharmaceutical formulation for treating the skin disorder is a 5% imiquimod lotion.

227. The package of any one of claims 217-222, wherein said topical pharmaceutical formulation for treating the skin disorder is a 5% imiquimod gel.

228. The package of any one of claims 217-222, wherein said topical pharmaceutical formulation for treating the skin disorder is an imiquimod pressure sensitive adhesive coating

229. The package of any one of claims 217-222, wherein said topical pharmaceutical formulation for treating the skin disorder is an imiquimod adhesive-coated sheet material.

230. The package of any one of claims 217-222, wherein said topical pharmaceutical formulation for treating the skin disorder is a 5% imiquimod foam.

231. The package of any one of claims 217-230, wherein the skin disorder is molluscum contagiosum.

232. The package of any one of claims 217-231, wherein the skin disorder is selected from a group consisting of genital warts, perianal warts and condyloma acuminata.

233. The package of any one of claims 217-232, wherein the skin disorder is actinic keratosis.

234. The package of any one of claims 217-233, wherein the skin disorder is superficial basal cell carcinoma.

235. The package of any one of claims 217-234, wherein the information is provided in a package drug insert.

236. The package of any one of claims 217-235, wherein the information is provided in a label for imiquimod approved by the FDA.

237. The package of any one of claims 217-236, wherein the child is between about 2 and about 12 years of age.

238. A package for enhancing the safety of using imiquimod as topical therapy for treating a skin disorder comprising:
(a) a topical pharmaceutical formulation containing imiquimod for treating the skin disorder; and
(b) information advsing that the imiquimod topical pharmaceutical formulation may cause a median multiple-dose peak serum imiquimod level of less than about 0.2 ng/ml in a child.

239. The package of claim 238, wherein said information further includes a warning that a decrease in the child's median white blood cell count or a decrease in the child's absolute neutrophil count is a side effect of using imiquimod to treat the skin disorder.

240. The package of any one of claims 238 or 239, wherein said information further includes information that a median multiple-dose peak serum imiquimod level of less than about 0.2 ng/ml is a side effect of using imiquimod.

241. The package of any one of claims 238-240, wherein said information further includes information that a median multiple-dose peak serum imiquimod level of about 0.2 or about 0.5 ng/ml is a side effect of using the imiquimod.

242. The package of any one of claims 238-241, wherein said information further includes information that a median multiple-dose peak serum imiquimod level selected from the group of about 0.1, about 0.15 ng/ml and about 0.3 ng/ml is a side effect of using imiquimod.

243. The package of any one of claims 238-242, wherein said information further includes information that a decrease in the child's median white blood cell count by about 1.4*109/L is a side effect of using imiquimod.

244. The package of any one of claims 238-243, wherein said information further includes information that a decrease in the child's median absolute neutrophil count decreased by 1.42*10⁹/L is a side effect of using imiquimod.

245. The package of any one of claims 238-244, wherein said topical pharmaceutical formulation for treating the skin disorder is a 5% imiquimod cream.

246. The package of any one of claims 238-244, wherein said topical pharmaceutical formulation for treating the skin disorder is a 5% imiquimod ointment.

247. The package of any one of claims 238-244, wherein said topical pharmaceutical formulation for treating the skin disorder is a 5% imiquimod salve.

248. The package of any one of claims 238-244, wherein said topical pharmaceutical formulation for treating the skin disorder is a 5% imiquimod lotion.

249. The package of any one of claims 238-244, wherein said topical pharmaceutical formulation for treating the skin disorder is a 5% imiquimod gel.

250. The package of any one of claims 238-244, wherein said topical pharmaceutical formulation for treating the skin disorder is an imiquimod pressure sensitive adhesive coating.

251. The package of any one of claims 238-244, wherein said topical pharmaceutical formulation for treating the skin disorder is an imiquimod adhesive-coated sheet material.

252. The package of any one of claims 238-244, wherein said topical pharmaceutical formulation for treating the skin disorder is a 5% imiquimod foam.

253. The package of any one of claims 238-252, wherein the skin disorder is molluscum contagiosum.

254. The package of any one of claims 238-253, wherein the skin disorder is selected from a group consisting of genital warts, perianal warts and condyloma acuminata.

255. The package of any one of claims 238-254, wherein the skin disorder is actinic keratosis.

256. The package of any one of claims 235-255, wherein the skin disorder is superficial basal cell carcinoma.

257. The package of any one of claims 238-256, wherein the information is provided in a package drug insert.

258. The package of any one of claims 238-256, wherein the information is provided in a label for imiquimod approved by the FDA.

259. The package of any one of claims 238-258, wherein the child is between about 2 and about 12 years of age.

260. A package for enhancing the safety of using imiquimod as topical therapy for treating a skin disorder comprising:
(a) a topical pharmaceutical formulation containing imiquimod for treating the skin disorder; and
(b) information containing a warning that the imiquimod topical pharmaceutical formulation may cause a median multiple-dose peak serum imiquimod level of less than median multiple-dose peak serum imiquimod level of about 0.2 or about 0.5 ng/ml in a child.

261. The package of claim 261, said information further includes a warning that a decrease in the child's median white blood cell count or a decrease in the child's absolute neutrophil count is a side effect of using imiquimod to treat the skin disorder.

262. The package of any one of claims 260-261, wherein said information further includes a warning that a median multiple-dose peak serum imiquimod level of less than about 0.2 ng/ml is a side effect of using imiquimod.

263. The package of any one of claims 260-262, wherein said information further includes a warning that a median multiple-dose peak serum imiquimod level of about 0.2 or about 0.5 ng/ml is a side effect of using the imiquimod.

264. The package of any one of claims 260-263, wherein said information further includes a warning that a median multiple-dose peak serum imiquimod level selected from the group of about 0.1, about 0.15 ng/ml and about 0.3 ng/ml is a side effect of using imiquimod.

265. The package of any one of claims 260-264, wherein said information further includes a warning that a decrease in the child's median white blood cell count by about 1.4*109/L is a side effect of using imiquimod.

266. The package of any one of claims 260-265, wherein said information further includes a warning that a decrease in the child's median absolute neutrophil count by 1.42*10⁹/L is a side effect of using imiquimod.

267. The package of any one of claims 260-266, wherein said topical pharmaceutical formulation for treating the skin disorder is a 5% imiquimod cream.

268. The package of any one of claims 260-266, wherein said topical pharmaceutical formulation for treating the skin disorder is a 5% imiquimod ointment.

269. The package of any one of claims 260-266, wherein said topical pharmaceutical formulation for treating the skin disorder is a 5% imiquimod salve.

270. The package of any one of claims 260-266, wherein said topical pharmaceutical formulation for treating the skin disorder is a 5% imiquimod lotion.

271. The package of any one of claims 260-266, wherein said topical pharmaceutical formulation for treating the skin disorder is a 5% imiquimod gel.

272. The package of any one of claims 260-266, wherein said topical pharmaceutical formulation for treating the skin disorder is an imiquimod pressure sensitive adhesive coating

273. The package of any one of claims 260-266, wherein said topical pharmaceutical formulation for treating the skin disorder is an imiquimod adhesive-coated sheet material.

274. The package of any one of claims 260-266, wherein said topical pharmaceutical formulation for treating the skin disorder is a 5% imiquimod foam.

275. The package of any one of claims 260-274, wherein the skin disorder is molluscum contagiosum.

276. The package of any one of claims 260-275, wherein the skin disorder is selected from a group consisting of genital warts, perianal warts and condyloma acuminata.

277. The package of any one of claims 260-276, wherein the skin disorder is actinic keratosis.

278. The package of any one of claims 260-277, wherein the skin disorder is superficial basal cell carcinoma.

279. The package of any one of claims 260-278, wherein the warning is provided in a package drug insert.

280. The package of any one of claims 260-278, wherein the warning is provided in a label for imiquimod approved by the FDA.

281. The package of any one of claims 260-280, wherein the child is between about 2 and about 12 years of age.

282. The package of any one of claims 260-280, wherein the child is between about 2 and about 5 years of age.

283. A package for enhancing the safety of using imiquimod as topical therapy for treating a skin disorder comprising:
(a) a topical pharmaceutical formulation containing imiquimod for treating the skin disorder; and
(b) information providing a warning that the imiquimod topical pharmaceutical formulation may cause a median multiple-dose peak serum imiquimod level, selected from the group of about 0.1, about 0.15 ng/ml and about 0.3 ng/ml, in a child.

284. The package of claim 283, wherein said information further includes a warning that a median multiple-dose peak serum imiquimod level, selected from the group of about 0.1, about 0.15 ng/ml and about 0.3 ng/ml, is a side effect of using imiquimod.

285. The package of any one of claims 283 or 284, wherein said information further includes a warning that a decrease in the child's median white blood cell count or a decrease in the child's absolute neutrophil count is a side effect of using imiquimod to treat the skin disorder.

286. The package of any one of claims 283-285, wherein said information further includes a warning that a median multiple-dose peak serum imiquimod level of less than about 0.2 ng/ml is a side effect of using imiquimod.

287. The package of any one of claims 283-286, wherein said information further includes a warning that a median multiple-dose peak serum imiquimod level of about 0.2 or about 0.5 ng/ml is a side effect of using the imiquimod.

288. The package of any one of claims 283-287, wherein said information further includes a warning that a median multiple-dose peak serum imiquimod level selected from the group of about 0.1, about 0.15 ng/ml and about 0.3 ng/ml is a side effect of using imiquimod.

289. The package of any one of claims 283-288, wherein said information further includes a warning that a decrease in the child's median white blood cell count by about 1.4*109/L is a side effect of using imiquimod.

290. The package of any one of claims 283-289, wherein said information further includes a warning that a decrease in the child's median absolute neutrophil count by 1.42*10⁹/L is a side effect of using imiquimod.

291. The package of any one of claims 283-290, wherein said topical pharmaceutical formulation for treating the skin disorder is a 5% imiquimod cream.

292. The package of any one of claims 283-290, wherein said topical pharmaceutical formulation for treating the skin disorder is a 5% imiquimod ointment.

293. The package of any one of claims 283-290, wherein said topical pharmaceutical formulation for treating the skin disorder is a 5% imiquimod salve.

294. The package of any one of claims 283-290, wherein said topical pharmaceutical formulation for treating the skin disorder is a 5% imiquimod lotion.

295. The package of any one of claims 283-290, wherein said topical pharmaceutical formulation for treating the skin disorder is a 5% imiquimod gel.

296. The package of any one of claims 283-290, wherein said topical pharmaceutical formulation for treating the skin disorder is an imiquimod pressure sensitive adhesive coating

297. The package of any one of claims 283-290, wherein said topical pharmaceutical formulation for treating the skin disorder is an imiquimod adhesive-coated sheet material.

298. The package of any one of claims 283-290, wherein said topical pharmaceutical formulation for treating the skin disorder is a 5% imiquimod foam.

299. The package of any one of claims 283-298, wherein the skin disorder is molluscum contagiosum.

300. The package of any one of claims 283-299, wherein the skin disorder is selected from a group consisting of genital warts, perianal warts and condyloma acuminata.

301. The package of any one of claims 283-300, wherein the skin disorder is actinic keratosis.

302. The package of any one of claims 283-301, wherein the skin disorder is superficial basal cell carcinoma.

303. The package of any one of claims 283-302, wherein the warning is provided in a package drug insert.

304. The package of any one of claims 283-303, wherein the warning is provided in a label for imiquimod approved by the FDA.

305. The package of any one of claims 283-304, wherein the child is between about 6 and about 12 years of age.

306. A method of using imiquimod as topical therapy for treating a child diagnosed with a skin disorder comprising:
(a) topically administering a therapeutically effective amount of imiquimod to a child in need of treatment;
(b) monitoring the child for the appearance of at least one symptom of systemic absorption of imiquimod; and
(c) if at least one symptom of systemic absorption of imiquimod is observed, reducing or tapering off the dosage of the imiquimod to a dosage that does not produce the at least one symptom of systemic absorption.

307. The method of claim 306, wherein if at least one symptom of systemic absorption of imiquimod is observed, administration of imiquimod is discontinued.

308. The method of any one of claims 306 or 307, wherein if at least one symptom of systemic absorption of imiquimod is observed, the patient is further tested for a decrease in mean white blood cell count or a decrease in median absolute neutrophil count.

309. The method of claim 308, wherein the testing comprises clinical blood tests.

310. The method of any one of claims 306-309, further comprising administering a therapeutically effective amount of imiquimod after at least one symptom of systemic absorption has subsided or returned to normal.

311. The method of any one of claims 306-310, wherein the therapeutically effective dose amount of imiquimod is about 12.5 mg, about 25 mg, or about 37.5 mg.

312. The method of any one of claims 306-311, wherein the therapeutically effective amount of imiquimod is provided in unit dose form.

313. The method of any one of claims 306-312, wherein the therapeutically effective dose amount of imiquimod is provided in a unit dose form or in multiple doses to provide for a course of topical imiquimod therapy.

314. The method of any one of claims 306-313, wherein said method includes the further step of providing information that a decrease in the child's median absolute neutrophil count is a potential sign of systemic absorption of imiquimod.

315. The method of any one of claims 306-308, wherein said method includes the further step of providing information that a decrease in the child's median absolute neutrophil count is a sign of systemic absorption of imiquimod.

316. The method of any one of claims 306-315, wherein said method includes the further step of providing information that a decrease in the child's median absolute neutrophil count is a side effect of using the imiquimod topical pharmaceutical formulation.

317. The method of any one of claims 306-316, wherein said method includes the further step of providing information that the skin disorder is molluscum contagiosum.

318. The method of any one of claims 3056-317, wherein said method includes the further step of providing information that the skin disorder is selected from a group consisting of genital warts, perianal warts and condyloma acuminata.

319. The method of any one of claims 306-318, wherein said method includes the further step of providing information that the skin disorder is actinic keratosis.

320. The method of any one of claims 306-319, wherein said method includes the further step of providing information that the skin disorder is superficial basal cell carcinoma.

321. The method of any one of claims 306-320, wherein said method includes the further step of providing information that the child is of an age of between about 2 and about 12 years old.

322. The method of any one of claims 305-320, wherein said method includes the further step of providing information that the child is of an age of between about 6 and about 12 years old.

323. The method of any one of claims 306-320, wherein said method includes the further step of providing information that the child is of an age of between about 2 and about 5 years old.

324. The method of any one of claims 306-323, wherein said method includes the further step of providing information that the median absolute neutrophil count decreased by about 1.42*10⁹/L.

325. The method of any one of claims 306-324, wherein said method further includes the step of providing information that the imiquimod is provided as an imiquimod 5% cream for topical use.

326. The package of any one of claims 306-324, wherein said method further includes the step of providing information that the imiquimod is provided as a 5% imiquimod ointment.

327. The package of any one of claims 306-324, wherein said method further includes the step of providing information that the imiquimod is provided as a 5% imiquimod salve.

328. The package of any one of claims 306-324, wherein said method further includes the step of providing information that the imiquimod is provided as a 5% imiquimod lotion.

329. The package of any one of claims 306-324, wherein said method further includes the step of providing information that the imiquimod is provided as a 5% imiquimod gel.

330. The package of any one of claims 306-324, wherein said method further includes the step of providing information that the imiquimod is provided as an imiquimod pressure sensitive adhesive coating

331. The package of any one of claims 306-324, wherein said method further includes the step of providing information that the imiquimod is provided as an imiquimod adhesive-coated sheet material.

332. The package of any one of claims 306-324, wherein said method further includes the step of providing information that the imiquimod is provided as a 5% imiquimod foam.

333. The method of any one of claims 306-332, wherein the information is provided in a package drug insert.

334. The method of any one of claims 306-332, wherein the information is provided in a label for imiquimod approved by the FDA.

335. A method of administering imiquimod as a topical therapy for a child diagnosed with a skin disorder comprising:
(a) providing the child with a therapeutically effective amount of imiquimod; and
(b) informing the child, a guardian of the child or a nurse or physician treating the child that a serum level of imiquimod, a decrease in median white blood cell count or a decrease in median absolute neutrophil count are symptoms of imiquimod systemic absorption which may require prompt medical evaluation if such symptoms are observed or if the peak serum imiquimod concentration exceeds more than about 2 ng/mL in the child.

336. The method of claim 335, wherein the child, the guardian of the child or the nurse or physician treating the child are informed by reference to a package drug insert.

337. The method of any one of claims 335 or 336, wherein the child, the guardian of the child or the nurse or physician treating the child are informed by a label approved by the FDA for imiquimod.

338. The method of any one of claims 335-337, wherein said method includes the further step of providing information that the skin disorder is molluscum contagiosum.

339. The method of any one of claims 335-338, wherein said method includes the further step of providing information that the skin disorder is selected from a group consisting of genital warts, perianal warts and condyloma acuminata.

340. The method of any one of claims 335-339, wherein said method includes the further step of providing information that the skin disorder is actinic keratosis.

341. The method of any one of claims 335-340, wherein said method includes the further step of providing information that the skin disorder is superficial basal cell carcinoma.

342. The method of any one of claims 335-341, wherein said method includes the further step of providing information that the child is of an age of between about 2 and about 12 years old.

343. The method of any one of claims 335-342, wherein said method includes the further step of providing information that the child is of an age of between about 6 and about 12 years old.

344. The method of any one of claims 335-343, wherein said method includes the further step of providing information that the child is of an age of between about 2 and about 5 years old.

345. The method of any one of claims 335-344, wherein said method includes the further step of providing information that the median absolute neutrophil count decreased by about 1.42*10⁹/L.

346. The method of any one of claims 335-345, wherein said method further includes the step of providing information that the imiquimod is provided as an imiquimod 5% cream for topical use.

347. The package of any one of claims 335-345, wherein said method further includes the step of providing information that the imiquimod is provided as a 5% imiquimod ointment.

348. The package of any one of claims 335-345, wherein said method further includes the step of providing information that the imiquimod is provided as a 5% imiquimod salve.

349. The package of any one of claims 335-345, wherein said method further includes the step of providing information that the imiquimod is provided as a 5% imiquimod lotion.

350. The package of any one of claims 335-345, wherein said method further includes the step of providing information that the imiquimod is provided as a 5% imiquimod gel.

351. The package of any one of claims 335-345, wherein said method further includes the step of providing information that the imiquimod is provided as an imiquimod pressure sensitive adhesive coating

352. The package of any one of claims 335-345, wherein said method further includes the step of providing information that the imiquimod is provided as an imiquimod adhesive-coated sheet material.

353. The package of any one of claims 335-345, wherein said method further includes the step of providing information that the imiquimod is provided as a 5% imiquimod foam.

354. The method of any one of claims 338-353, wherein said method further includes the step of providing information in a package drug insert.

355. The method of any one of claims 338-353, wherein said method further includes the step of providing information in a label for imiquimod approved by the FDA.

356. A method of using imiquimod as a topical therapy for a child diagnosed with a skin disorder comprising:
(a) advising a physician prescribing imiquimod to the child to monitor the child for one or more symptoms selected from a group consisting of a serum level of imiquimod, a decrease in median white blood cell count or a decrease in median absolute neutrophil count;
(b) recommending that when one or more of the symptoms is observed, an appropriate diagnostic be employed by the physician to determine whether one or more of the symptoms are present; and
(c) recommending that the physician remove, reduce, or taper off imiquimod dosing in the child until the serum level of the child returns to normal or does not exceed a peak serum imiquimod concentration of more than about 2 ng/mL.

357. The method of claim 356, wherein the physician is informed by reference to a package drug insert.

358. The method of claim 356, wherein the physician is informed by a label approved by the FDA for imiquimod.

359. The method of any one of claims 356-358, wherein said method includes the further step of providing information that the skin disorder is molluscum contagiosum.

360. The method of any one of claims 356-359, wherein said method includes the further step of providing information that the skin disorder is selected from a group consisting of genital warts, perianal warts and condyloma acuminata.

361. The method of any one of claims 356-360, wherein said method includes the further step of providing information that the skin disorder is actinic keratosis.

362. The method of any one of claims 356-361, wherein said method includes the further step of providing information that the skin disorder is superficial basal cell carcinoma.

363. The method of any one of claims 356-362, wherein said method includes the further step of providing information that the child is of an age of between about 2 and about 12 years old.

364. The method of any one of claims 356-363, wherein said method includes the further step of providing information that the child is of an age of between about 6 and about 12 years old.

365. The method of any one of claims 356-364, wherein said method includes the further step of providing information that the child is of an age of between about 2 and about 5 years old.

366. The method of any one of claims 365-365, wherein said method includes the further step of providing information that the median absolute neutrophil count decreased by about 1.42*10⁹/L.

367. The method of any one of claims 356-365, wherein said method further includes the step of providing information that the imiquimod is provided as an imiquimod 5% cream for topical use.

368. The method of any one of claims 356-366, wherein said method further includes the step of providing information that the imiquimod is provided as a 5% imiquimod ointment.

369. The method of any one of claims 356-366, wherein said method further includes the step of providing information that the imiquimod is provided as a 5% imiquimod salve.

370. The method of any one of claims 356-366, wherein said method further includes the step of providing information that the imiquimod is provided as a 5% imiquimod lotion.

371. The method of any one of claims 356-366, wherein said method further includes the step of providing information that the imiquimod is provided as a 5% imiquimod gel.

372. The method of any one of claims 356-366, wherein said method further includes the step of providing information that the imiquimod is provided as an imiquimod pressure sensitive adhesive coating

373. The method of any one of claims 356-366, wherein said method further includes the step of providing information that the imiquimod is provided as an imiquimod adhesive-coated sheet material.

374. The method of any one of claims 356-366, wherein said method further includes the step of providing information that the imiquimod is provided as a 5% imiquimod foam.

375. The method of any one of claims 359-374, wherein said method further includes the step of providing information in a package drug insert.

376. The method of any one of claims 359-374, wherein said method further includes the step of providing information in a label for imiquimod approved by the FDA.

377. A method for using imiquimod as topical therapy for treating a child diagnosed with a skin disorder comprising:
(a) monitoring a patient who is receiving administrations of imiquimod for one or more symptoms; and
(b) if one or more of said symptoms selected from a group consisting of a serum level of imiquimod that exceeds a peak serum imiquimod concentration of more than about 2 ng/mL, a decrease in median white blood cell count by about 1.4*109/L or a decrease in median absolute neutrophil count by about 1.42*10⁹/L are observed, reducing or tapering off the imiquimod dosing until the child's symptoms have subsided.

378. The method of claim 377, wherein the imiquimod dosing is increased after the child's symptoms have subsided.

379. The method of any one of claims 377 or 378, wherein a physician who is treating the child is informed by reference to a package drug insert.

380. The method of claim 379, wherein a physician who is treating the child is informed by a label approved by the FDA for imiquimod.

381. The method of any one of claims 377-380, wherein said method includes the further step of providing information that the skin disorder is molluscum contagiosum.

382. The method of any one of claims 377-380, wherein said method includes the further step of providing information that the skin disorder is selected from a group consisting of genital warts, perianal warts and condyloma acuminata.

383. The method of any one of claims 377-380, wherein said method includes the further step of providing information that the skin disorder is actinic keratosis.

384. The method of any one of claims 377-380, wherein said method includes the further step of providing information that the skin disorder is superficial basal cell carcinoma.

385. The method of any one of claims 377-384, wherein said method includes the further step of providing information that the child is of an age of between about 2 and about 12 years old.

386. The method of any one of claims 377-384, wherein said method includes the further step of providing information that the child is of an age of between about 6 and about 12 years old.

387. The method of any one of claims 377-384, wherein said method includes the further step of providing information that the child is of an age of between about 2 and about 5 years old.

388. The method of any one of claims 377-387, wherein said method further includes the step of providing information that the imiquimod is provided as an imiquimod 5% cream for topical use.

389. The method of any one of claims 377-387, wherein said method further includes the step of providing information that the imiquimod is provided as a 5% imiquimod ointment.

390. The method of any one of claims 377-387, wherein said method further includes the step of providing information that the imiquimod is provided as a 5% imiquimod salve.

391. The method of any one of claims 377-387, wherein said method further includes the step of providing information that the imiquimod is provided as a 5% imiquimod lotion.

392. The method of any one of claims 377-387, wherein said method further includes the step of providing information that the imiquimod is provided as a 5% imiquimod gel.

393. The package of any one of claims 377-387, wherein said method further includes the step of providing information that the imiquimod is provided as an imiquimod pressure sensitive adhesive coating

394. The method of any one of claims 377-387, wherein said method further includes the step of providing information that the imiquimod is provided as an imiquimod adhesive-coated sheet material.

395. The method of any one of claims 377-387, wherein said method further includes the step of providing information that the imiquimod is provided as a 5% imiquimod foam.

396. The method of any one of claims 380-395, wherein said method further includes the step of providing information in a package drug insert.

397. The method of any one of claims 380-395, wherein said method further includes the step of providing information in a label for imiquimod approved by the FDA.

398. A method of using imiquimod as topical therapy for a child diagnosed with a skin disorder that is prescribed by a physician comprising:
(a) monitoring the child who is receiving administrations of imiquimod for one or more symptoms selected from a group consisting of a serum level of imiquimod that exceeds a peak serum imiquimod concentration of more than about 2 ng/mL, a decrease in median white blood cell count by about 1.4*109/L or a decrease in median absolute neutrophil count by about 1.42*10⁹/L;
(b) if one or more of the symptoms are observed, ceasing the imiquimod dosing until the symptoms have subsided.

399. The method of claim 398, wherein the imiquimod dosing is restored after the child's symptoms have subsided.

400. The method of any one of claims 398 or 399, wherein a physician who is treating the child is informed by reference to a package drug insert.

401. The method of any one of claims 398 or 399, wherein a physician who is treating the child is informed by a label approved by the FDA for imiquimod.

402. The method of any one of claims 398-401, wherein said method includes the further step of providing information that the skin disorder is molluscum contagiosum.

403. The method of any one of claims 398-401, wherein said method includes the further step of providing information that the skin disorder is selected from a group consisting of genital warts, perianal warts and condyloma acuminata.

404. The method of any one of claims 398-401, wherein said method includes the further step of providing information that the skin disorder is actinic keratosis.

405. The method of any one of claims 398-401, wherein said method includes the further step of providing information that the skin disorder is superficial basal cell carcinoma.

406. The method of any one of claims 398-405, wherein said method includes the further step of providing information that the child is of an age of between about 2 and about 12 years old.

407. The method of any one of claims 398-405, wherein said method includes the further step of providing information that the child is of an age of between about 6 and about 12 years old.

408. The method of any one of claims 398-405, wherein said method includes the further step of providing information that the child is of an age of between about 2 and about 5 years old.

409. The method of any one of claims 398-408, wherein said method further includes the step of providing information that the imiquimod is provided as an imiquimod 5% cream for topical use.

410. The method of any one of claims 398-408, wherein said method further includes the step of providing information that the imiquimod is provided as a 5% imiquimod ointment.

411. The method of any one of claims 398-408, wherein said method further includes the step of providing information that the imiquimod is provided as a 5% imiquimod salve.

412. The method of any one of claims 398-408, wherein said method further includes the step of providing information that the imiquimod is provided as a 5% imiquimod lotion.

413. The method of any one of claims 398-408, wherein said method further includes the step of providing information that the imiquimod is provided as a 5% imiquimod gel.

414. The package of any one of claims 398-408, wherein said method further includes the step of providing information that the imiquimod is provided as an imiquimod pressure sensitive adhesive coating

415. The method of any one of claims 398-408, wherein said method further includes the step of providing information that the imiquimod is provided as an imiquimod adhesive-coated sheet material.

416. The method of any one of claims 398-408, wherein said method further includes the step of providing information that the imiquimod is provided as a 5% imiquimod foam.

417. The method of any one of claims 401-416, wherein said method further includes the step of providing information in a package drug insert

418. The method of any one of claims 401-416, wherein said method further includes the step of providing information in a label for imiquimod approved by the FDA.

419. An enhanced safety method of using imiquimod as therapy for treating an area of skin of a child affected with a skin disorder molluscum contagiosum, comprising:
(a) providing the child, a guardian of the child, or nurse or physician treating the child, with a therapeutically effective amount of imiquimod for application to an area of the child's skin affected with the molluscum contagiosum;
(b) providing the child, the guardian of the child, or the nurse or the physician treating the child, with information on how to apply the therapeutically effective amount of imiquimod to the area of the child's skin affected with the molluscum contagiosum,
(c) providing the child, the guardian of the child or the nurse or the physician treating the child with information that systemic absorption of imiquimod across the affected skin may be observed during the therapy;
(d) providing the child, the guardian of the child or the nurse or the physician treating the child with information that medical evaluation may be required if systemic absorption of imiquimod is experienced by the child during the therapy.

420. A method of claim 419, including the further step of providing packaging that includes the therapeutically effective amount of imiquimod in a pharmaceutical formulation.

421. A method of any one of claims 419-420, wherein the pharmaceutical formulation is a topical formulation.

422. The method of any one of claims 419-421, wherein said method includes the further step of providing information that the skin disorder is molluscum contagiosum.

423. The method of any one of claims 419-422, wherein said method includes the further step of providing information that the skin disorder further includes a disorder selected from a group consisting of genital warts, perianal warts and condyloma acuminata.

424. The method of any one of claims 419-423, wherein said method includes the further step of providing information that the skin disorder also includes actinic keratosis.

425. The method of any one of claims 419-424, wherein said method includes the further step of providing information that the skin disorder further includes superficial basal cell carcinoma.

426. The method of any one of claims 419-425, wherein said method includes the further step of providing information that the child is of an age of between about 2 and about 12 years old.

427. The method of any one of claims 419-426, wherein said method includes the further step of providing information that the child is of an age of between about 6 and about 12 years old.

428. The method of any one of claims 419-427, wherein said method includes the further step of providing information that the child is of an age of between about 2 and about 5 years old.

429. The method of any one of claims 419-428, wherein said method includes the further step of providing information that the median absolute neutrophil count decreased by about 1.42*10⁹/L.

430. The method of any one of claims 419-429, wherein said method further includes the step of providing information that the imiquimod is provided as an imiquimod 5% cream for topical use.

431. The method of any one of claims 419-429, wherein said method further includes the step of providing information that the imiquimod is provided as a 5% imiquimod ointment.

432. The method of any one of claims 419-429, wherein said method further includes the step of providing information that the imiquimod is provided as a 5% imiquimod salve.

433. The method of any one of claims 419-429, wherein said method further includes the step of providing information that the imiquimod is provided as a 5% imiquimod lotion.

434. The method of any one of claims 419-429, wherein said method further includes the step of providing information that the imiquimod is provided as a 5% imiquimod gel.

435. The method of any one of claims 419-429, wherein said method further includes the step of providing information that the imiquimod is provided as an imiquimod pressure sensitive adhesive coating

436. The method of any one of claims 419-429, wherein said method further includes the step of providing information that the imiquimod is provided as an imiquimod adhesive-coated sheet material.

437. The method of any one of claims 419-429, wherein said method further includes the step of providing information that the imiquimod is provided as a 5% imiquimod foam.

438. The method of any one of claims 422-437, wherein said method further includes the step of providing information in a package drug insert.

439. The method of any one of claims 422-437, wherein said method further includes the step of providing information in a label for imiquimod approved by the FDA.

440. An enhanced safety method of using imiquimod as therapy for treating an area of skin of a child affected with genital warts or perianal warts, comprising:
(a) providing the child, a guardian of the child, or a nurse or a physician treating the child, with a therapeutically effective amount of imiquimod for application to an area of the child's skin affected with the genital warts or perianal wart;
(b) providing the child, the guardian of the child, or the nurse or the physician treating the child, with information on how to apply the therapeutically effective amount of imiquimod to the area of the child's skin affected with the genital warts or perianal wart,
(c) providing the child, the guardian of the child, or the nurse or the physician treating the child with information that systemic absorption of imiquimod across the affected skin may be observed during the therapy;
(d) providing the child, the guardian of the child, or the nurse or the physician treating the child with information that medical evaluation may be required if systemic absorption of imiquimod is experienced by the child during the therapy.

441. A method of claim 440, including the further step of providing packaging that includes the therapeutically effective amount of imiquimod in a pharmaceutical formulation.

442. A method of any one of claims 440-441, wherein the pharmaceutical formulation is a topical formulation.

443. The method of any one of claims 440-442, wherein said method includes the further step of providing information that the skin disorder is also molluscum contagiosum.

444. The method of any one of claims 440-443T wherein said method includes the further step of providing information that the skin disorder is also a skin disorder selected from a group consisting of genital warts, perianal warts and condyloma acuminata.

445. The method of any one of claims 440-444, wherein said method includes the further step of providing information that the skin disorder is also actinic keratosis.

446. The method of any one of claims 440-445, wherein said method includes the further step of providing information that the skin disorder is also superficial basal cell carcinoma.

447. The method of any one of claims 440-446, wherein said method includes the further step of providing information that the child is of an age of between about 2 and about 12 years old.

448. The method of any one of claims 440-447, wherein said method includes the further step of providing information that the child is of an age of between about 6 and about 12 years old.

449. The method of any one of claims 440-448, wherein said method includes the further step of providing information that the child is of an age of between about 2 and about 5 years old.

450. The method of any one of claims 440-449, wherein said method includes the further step of providing information that the median absolute neutrophil count decreased by about 1.42*10⁹/L.

451. The method of any one of claims 440-450, wherein said method further includes the step of providing information that the imiquimod is provided as an imiquimod 5% cream for topical use.

452. The method of any one of claims 440-450, wherein said method further includes the step of providing information that the imiquimod is provided as a 5% imiquimod ointment.

453. The method of any one of claims 440-450, wherein said method further includes the step of providing information that the imiquimod is provided as a 5% imiquimod salve.

454. The method of any one of claims 440-450, wherein said method further includes the step of providing information that the imiquimod is provided as a 5% imiquimod lotion.

455. The method of any one of claims 440-450, wherein said method further includes the step of providing information that the imiquimod is provided as a 5% imiquimod gel.

456. The method of any one of claims 440-450, wherein said method further includes the step of providing information that the imiquimod is provided as an imiquimod pressure sensitive adhesive coating

457. The method of any one of claims 440-450, wherein said method further includes the step of providing information that the imiquimod is provided as an imiquimod adhesive-coated sheet material.

458. The method of any one of claims 440-450, wherein said method further includes the step of providing information that the imiquimod is provided as a 5% imiquimod foam.

459. The method of any one of claims 443-458, wherein said method further includes the step of providing information in a package drug insert.

460. The method of any one of claims 443-458, wherein said method further includes the step of providing information in a label for imiquimod approved by the FDA.

461. An enhanced safety method of using imiquimod as therapy for treating an area of skin of a child affected with actinic keratosis, comprising:
(a) providing the child, a guardian of the child or a nurse or the physician treating the child, with a therapeutically effective amount of imiquimod for application to an area of the child's skin affected with the actinic keratosis;
(b) providing the child, the guardian of the child, or the nurse or the physician treating the child, with information on how to apply the therapeutically effective amount of imiquimod to the area of the child's skin affected with the actinic keratosis,
(c) providing the child, the guardian of the child, or the nurse or the physician treating the child with information that systemic absorption of imiquimod across the affected skin may be observed during the therapy;
(d) providing the child, the guardian of the child, or the nurse or the physician treating the child with information that medical evaluation may be required if systemic absorption of imiquimod is experienced by the child during the therapy.

462. A method of claim 461, including the further step of providing packaging that includes the therapeutically effective amount of imiquimod in a pharmaceutical formulation.

463. A method of any one of claims 461 or 462, wherein the pharmaceutical formulation is a topical formulation.

464. The method of any one of claims 461-463, wherein said method includes the further step of providing information that the skin disorder is also molluscum contagiosum.

465. The method of any one of claims 461-464, wherein said method includes the further step of providing information that the skin disorder is also a skin disorder selected from a group consisting of genital warts, perianal warts and condyloma acuminata.

466. The method of any one of claims 461-465, wherein said method includes the further step of providing information that the skin disorder is actinic keratosis.

467. The method of any one of claims 461-466, wherein said method includes the further step of providing information that the skin disorder is also a superficial basal cell carcinoma.

468. The method of any one of claims 461-467, wherein said method includes the further step of providing information that the child is of an age of between about 2 and about 12 years old.

469. The method of any one of claims 461-468, wherein said method includes the further step of providing information that the child is of an age of between about 6 and about 12 years old.

470. The method of any one of claims 461-469, wherein said method includes the further step of providing information that the child is of an age of between about 2 and about 5 years old.

471. The method of any one of claims 461-470, wherein said method includes the further step of providing information that the median absolute neutrophil count decreased by about 1.42*10⁹/L.

472. The method of any one of claims 461-471, wherein said method further includes the step of providing information that the imiquimod is provided as an imiquimod 5% cream for topical use.

473. The method of any one of claims 461-471, wherein said method further includes the step of providing information that the imiquimod is provided as a 5% imiquimod ointment.

474. The method of any one of claims 461-471, wherein said method further includes the step of providing information that the imiquimod is provided as a 5% imiquimod salve.

475. The method of any one of claims 461-471, wherein said method further includes the step of providing information that the imiquimod is provided as a 5% imiquimod lotion.

476. The method of any one of claims 461-471, wherein said method further includes the step of providing information that the imiquimod is provided as a 5% imiquimod gel.

477. The method of any one of claims 461-471, wherein said method further includes the step of providing information that the imiquimod is provided as an imiquimod pressure sensitive adhesive coating

478. The method of any one of claims 461-471, wherein said method further includes the step of providing information that the imiquimod is provided as an imiquimod adhesive-coated sheet material.

479. The method of any one of claims 461-471, wherein said method further includes the step of providing information that the imiquimod is provided as a 5% imiquimod foam.

480. The method of any one of claims 464-479, wherein said method further includes the step of providing information in a package drug insert.

481. The method of any one of claims 464-479, wherein said method further includes the step of providing information in a label for imiquimod approved by the FDA.
